(19) United States
(12) Patent Application Publication
(10) Pub. No.: US 2010/0280041 A1
(43) Pub. Date: Nov. 4, 2010

(54) RHOKINASE-DEPENDENT INHIBITION ACTIVITY ON PULMONARY ARTERY ENDOTHELIUM DYSFUNCTION, MEDIAL WALL THICKNESS AND VASCULAR OBSTRUCTION OF PULMODIL AND PULMODIL-1

(75) Inventor: Ing-Jun Chen, Kaohsiung City (TW)

Correspondence Address: VOLPE AND KOENIG, P.C., UNITED PLAZA, 30 SOUTH 17TH STREET, PHILADELPHIA, PA, 19103, US

(73) Assignee: KAOHSIUNG MEDICAL UNIVERSITY, Kaohsiung City (TW)

(21) Appl. No.: 12/630,368

(22) Filed: Dec. 3, 2009

(30) Foreign Application Priority Data
Apr. 30, 2009 (TW) 098114474

(51) Int. Cl. A61K 31/52; A61P 9/00

(52) U.S. Cl. 514/252.16

(57) ABSTRACT

A pharmaceutical composition for treating one of a cardiovascular disease and a pulmonary artery disease, comprising one of a first compound having a Formula I and a second compound having a Formula II.

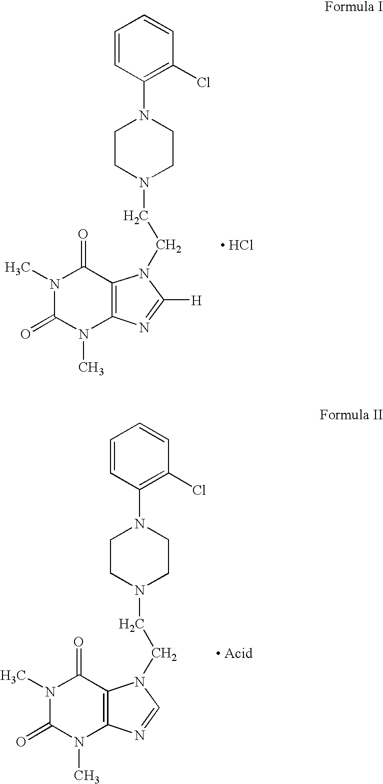

### FIELD OF THE INVENTION

The present invention relates to chlorophenylpiperazine salt derivatives, 7-[2-[4-(2-chlorobenzene)piperazinyl]ethyl]-1,3-dimethylxanthine.HCl (hereinafter referred to as “Pulmodil”) and 7-[2-[4-(2-chlorophenyl)piperazinyl]-ethyl]-1,3-dimethylxanthine. Citric acid (hereinafter referred to as “Pulmodil-1”), obtained by a recrystallization method, and particularly relates to the activities of inhibiting a pulmonary artery endothelium dysfunction, a thickened pulmonary artery medial wall, and a vascular obstruction thereof.

### BACKGROUND OF THE INVENTION

Pulmonary arterial hypertension (PAH) caused by the dysfunction of pulmonary artery is a progressive and lethal disease. Raised pulmonary arterial pressure and the remodeling of the pulmonary vessels may cause right ventricular hypertrophy or failure, which may increase the risk and the death rate of the patients. Before becoming a lethal disease, the dysfunction of pulmonary artery has many early symptoms, such as injury, propagation or hypercontractility of endothelium of vascular smooth muscle, a transfer of inflammatory cells, etc.

Endothelium of pulmonary vessels is a specific place where various vasoactive mediators changing tensions of oxygen and carbon dioxide in the blood, blood pressure and flow, etc are produced. One of a diastolic mediator, Nitric oxide (NO), is generated by the endothelial NO synthase (eNOS) and effective in relaxing smooth muscle, inhibiting the activations of Neutrophil and platelet and improving the proliferation of the smooth muscle. NO is capable of stimulating its target enzyme, soluble guanylyl cyclase (sGC), for increasing the secondary messenger, cyclic guanosine monophosphate (cGMP), in tissues. Consequently, the increased cGMP will activate protein kinase G (PKG), and the PKG further phosphorylates several proteins related to the regulations of calcium ions in cells for achieving the function of relaxing vessels.

In many complicated mechanisms related to a pulmonary artery endothelium dysfunction, a thickened pulmonary artery medial wall, and a vascular obstruction, it is indicated that the activation of rho-kinase is associated with the contraction of smooth muscle cells, actin cytoskeleton organization, cell division and gene expression. It is also proved by the rat and mice animal models and human patients with pulmonary artery endothelium dysfunction that the activation of rho-kinase is involved in the pathological characteristics of a pulmonary artery endothelium dysfunction, a thickened pulmonary artery medial wall, and a vascular obstruction, and the activated rho-kinase would increase the Ca²⁺ sensitivity of the vascular smooth muscle cell, and thus cause the vasoconstriction.

Since rho-kinase related study is increased recently, inhibiting the expression of rho-kinase has been developed to a method in treating vasoconstriction. For example, the developed rho-kinase inhibitor is used to relieve the pathological changes caused by the vascular tension.

The present invention provides a kind of compounds, such as Pulmodil and Pulmodil-1, effective in inhibiting the expression of rho-kinase, activating the expression of eNOS, and reducing the right ventricular hypertrophy caused by the hypoxia-induced PAH. According to the mechanism for the compound “Pulmodil”, it could be applied to the inhibition of a pulmonary artery endothelium dysfunction, a thickened pulmonary artery medial wall, a vascular obstruction and a cardiovascular disease. During the research process, the inventor found a simple synthetic method for the production of Pulmodil or Pulmodil-1. Pulmodil could be obtained from merely two reactants. Pulmodil has not only a good solubility, but also benefits of low toxicity and facile to be used in a therapeutical supplements. Therefore, the compound “Pulmodil” provided in the present invention is safe for a user in need thereof.

Hence, because of the defects in the prior arts, the inventors provide an observing device and method to effectively overcome the demerits existing in the prior arts.

### SUMMARY OF THE INVENTION

A chlorophenylpiperazine salt derivative, i.e. Pulmodil, which is obtained by reacting 2-chloroethyl theophylline with 2-chlorophenyl piperazine and then recrystallizing the intermediate therefrom, is provided in the present invention. Pulmodil has the activities of relieving a pulmonary artery endothelium dysfunction, a thickened pulmonary artery medial wall, and a vascular obstruction, and the benefits of good solubility, low toxicity and safety.

It is an aspect of the present invention, a pharmaceutical composition for treating one of a cardiovascular disease and a pulmonary artery disease is provided. The pharmaceutical composition comprises one of a first compound having a Formula I:

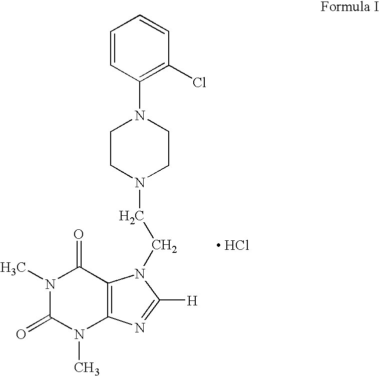

and a second compound having a Formula II:

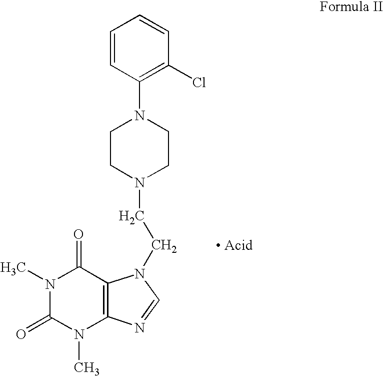

Preferably, the pulmonary artery disease comprises one selected from a group consisting of a pulmonary artery endothelium dysfunction, a thickened pulmonary artery medial wall, and a vascular obstruction.

Preferably, the HCl of Formula I and the acid of Formula II are derived from at least one of a xanthine and a piperazine.

Preferably, the acid is one of an organic acid and an inorganic acid.

Preferably, the organic acid comprises one selected from a group consisting of a citric acid, a maleinic acid, a fumaric acid, a tartaric acid, an oleic acid, a stearic acid, a benzenesulphonic acid, an ethyl benzenesulphonic acid, a benzoic acid, a succinic acid, a mesylic acid, a dimesylic acid, an acetic acid, a propionic acid, a pentanoic acid and an aspartic acid.

Preferably, the inorganic acid comprises one selected from a group consisting of a hydrochloride, a sulfuric acid, a phosphoric acid, a boric acid and a dihydrochloride.

Preferably, the pharmaceutical composition further comprises at least one of a pharmaceutically acceptable carrier and an excipient.

Preferably, the second compound is a 7-[2-[4-(2-chlorophenyl)piperazinyl]-ethyl]-1,3-dimethylxanthine. Citric acid.

In accordance with another aspect of the present invention, a method for relieving a symptom including one selected from a group consisting of a pulmonary artery endothelium dysfunction, a thickened pulmonary artery medial wall, and a vascular obstruction in a mammalian subject in need thereof is provided. The method comprises steps of administering to the mammalian subject a pharmaceutically effective amount of a pharmaceutical composition including one of a first compound having a Formula I:

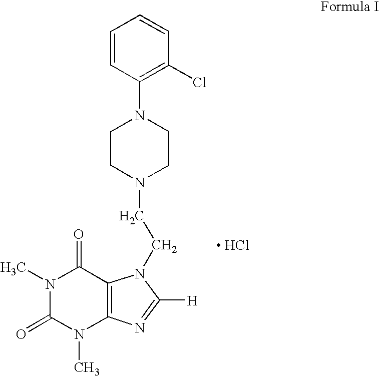

and a second compound having a Formula II:

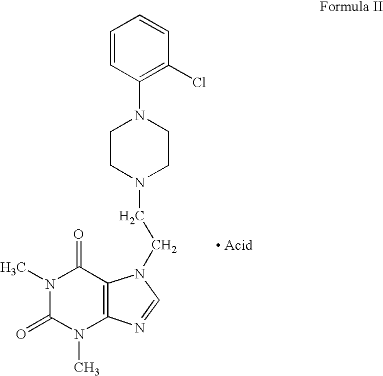

Preferably, the mammalian subject is a human.

Preferably, the administration comprises one selected from a group consisting of an oral administration, an intravenous injection, a subcutaneous injection, an intraperitoneal injection, an intramuscular injection and a sublingual administration.

Preferably, the pharmaceutical composition further comprises at least one of a pharmaceutically acceptable carrier and an excipient.

Preferably, the second compound is a 7-[2-[4-(2-chlorophenyl)piperazinyl]-ethyl]-1,3-dimethylxanthine. Citric acid.

In accordance with another aspect of the present invention, a method for treating a cardiovascular disease in a mammalian subject in need thereof is provided. The method comprises the steps of administering to the mammalian subject a pharmaceutically effective amount of a substrate including one of a first compound having a Formula I and a second compound having a Formula II.

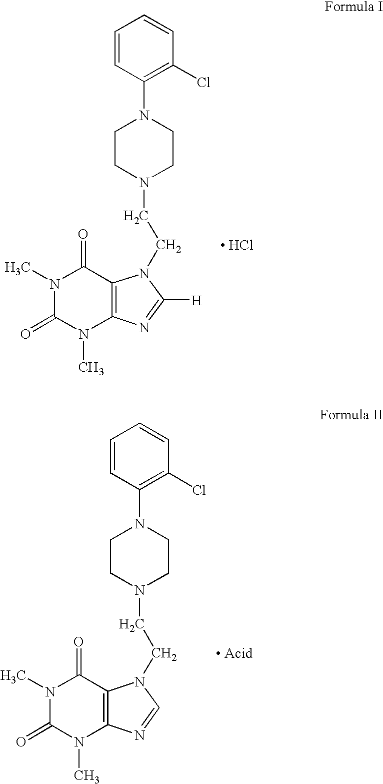

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIGS. 1 (A) and (B) are diagrams showing the structures of Pulmodil and Pulmodil-1;

FIGS. 2 (A) to (D) are diagrams respectively showing the comparison results between 5 ng/ml Pulmodil (A) and the standard (B) and between 200 ng/ml Pulmodil (C) and the standard (D);

FIGS. 3 (A) and (B) are diagrams showing the primary cultured TSMCs under (A) phase contrast microscope and (B) fluorescence microscope.;

FIGS. 4 (A) to (C) are diagrams showing the effects of different concentrations of Pulmodil, Pulmodil-1 and the major structural compound (MSC) on the survival rates of TSMCs of Wistar rat;

FIGS. 5 (A) to (C) are diagrams showing the effects of Pulmodil with different concentrations on the mean pulmonary arterial pressure (MPAP) in U46619-treated rats;

FIGS. 5 (D) and (E) are diagrams showing the effects of various drugs on the mean pulmonary arterial pressure (MPAP) in U46619-treated rats;

FIGS. 6 (A) to (C) are diagrams showing the effects of Pulmodil on phenylephrine and U46619 induced contractility of the pulmonary artery rings;

FIGS. 7 (A) and (B) are diagrams showing the effects of Pulmodil (A) and Y27632 (B) on K⁺-channel opening activity in the pulmonary artery smooth muscle cells;

FIGS. 8 (A) to (C) are diagrams showing the effects of Pulmodil and Y27632 on different drugs-induced Ca²⁺ mobilization;

FIG. 9 is a diagram showing the effects of Pulmodil on the smooth muscle contractions;

FIG. 10 is a diagram showing the effects of Pulmodil on the expressions of eNOS/sGC/PKG/PDE5A/ROCKII proteins in the U46619-treated rats;

FIGS. 11 (A) to (D) are diagrams showing the effects of Pulmodil on the expressions of eNOS/sGC/PKG/ROCKII proteins in the present of various inhibitors;

FIG. 12 is a diagram showing the effects of Pulmodil on the monocrotaline (MCT)-induced changes in expressions of eNOS/sGC/5-HTT/ROCKII proteins;

FIGS. 13 (A) and (B) are diagrams showing the effects of Pulmodil on the morphology of pulmonary artery (A) and right ventricle (B) in the MCT-treated rats;

FIGS. 14 (A) to (D) are diagrams showing the histological sections of pulmonary arteries of different groups of rats in Embodiment 4 ((A): group 1, (B): group 2, (C): group 3, (D): group 4);

FIG. 15 is a diagram showing the effects of Pulmodil on the pulmonary artery wall thickness of different groups of rats in Embodiment 4;

FIGS. 16 (A) and (B) are diagrams showing the morphometricly immunostaining for eNOS (A) and VEGF (B) in pulmonary arteries;

FIGS. 17 (A) to (D) are diagrams showing the histological sections of heart tissues of different groups of rats in Embodiment 4 ((A): group 1, (B): group 2, (C): group 3, (D): group 4);

FIG. 18 is a diagram showing the relative weight of right hart index of different groups of rats in Embodiment 4;

FIGS. 19 (A) to (G) are diagrams showing the effects of Pulmodil and Sildenafil on eNOS, sGC, PKG, PDE5A, ROCKII and VEGF protein expressions in different groups of rat lung tissue in Embodiment 4;

FIG. 20 is a diagram showing measurements of MPAP of different groups of rat in Embodiment 5;

FIGS. 21 (A) to (D) are diagrams showing the histological sections of pulmonary arteries of different groups of rats in Embodiment 5 ((A): group 1, (B): group 2, (C): group 3, (D): group 4);

FIGS. 22 (A) to (E) are diagrams showing the morphometricly immunostaining for eNOS in different groups of rat pulmonary arteries in Embodiment 5 ((A): group 1, (B): group 2, (C): negative control; (D): group 3, (E): group 4);

FIGS. 23 (A) and (B) are diagrams showing the right hart index represented by weight ratio (A) and area ratio (B) of different groups of rats in Embodiment 5;

FIGS. 24 (A) and (D) are diagrams the effects of Pulmodil and Sildenafil on eNOS, sGCa, PKG and ROCK II protein expressions in different groups of rat lung tissue in Embodiment 5;

FIGS. 25 (A) and (B) are diagrams showing the effects of Pulmodil or Sildenafil on ET-1 protein expression in lung tissue and ET-1 plasma concentration in different groups of rats in Embodiment 5; and

FIGS. 26 (A) and (B) are diagrams showing the concentration changes of Pulmodil in plasma in different groups of rats in Embodiment 5.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

A chlorophenylpiperazine salt derivative is provided in the present invention. The detailed descriptions of the preparation method, physical and chemical properties and the bioactivities of the chlorophenylpiperazine salt derivative are illustrated as follows.

### Embodiment 1

### Synthesis of Pulmodil

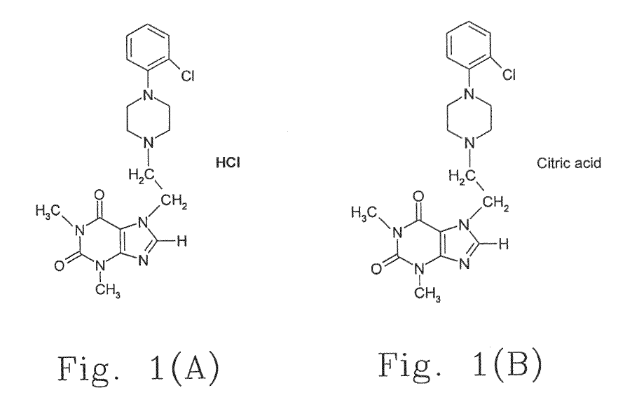

The first preferred embodiment of the present invention is Pulmodul. Method 1: 2-Chloroethyl theophylline, 2-chlorophenyl piperazine and sodium hydroxide (NaOH) (or sodium hydrogen carbonate, NaHCO₃) are dissolved in hydrous ethanol solution based on the molecular weight percentage and heated under reflux for three hours. After cooled overnight, the supernatant is decanted for proceeding the vacuum concentration and dry process, and then, one-fold volume of ethanol and three-fold volume of 2N hydrochloric acid (HCl) are added therein to dissolve at 50° C. to 60° C. as a saturated solution with pH 1.2. The saturated solution is sequentially decolorized with activated charcoal, filtered, deposited overnight and filtered to obtain a white crystal, i.e. Pulmodil. Pulmodil has a chemical formula as 7-[2-[4-(2-chlorobenzene)piperazinyl]ethyl]-1,3-dimethylxanthine.HCl, and a melting point of 249° C. to 252° C. The reaction formula is illustrated as follows. The chemical structure of Pulmodil is shown in FIG. 1 (A). Further, the purity of Pulmodil is determined with high performance liquid chromatography (HPLC).

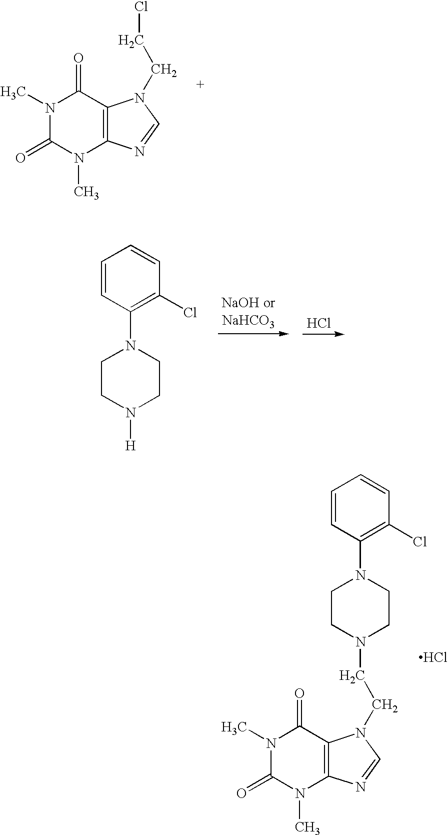

Method 2: 2-Chloroethyl theophylline and 2-chlorophenyl piperazine are dissolved in hydrous ethanol solution based on the molecular weight percentage and heated under reflux for three hours. After cooled overnight, the supernatant is decanted for proceeding the vacuum concentration and dry process, and then, one-fold volume of ethanol and three-fold volume of 2N hydrochloric acid (HCl) are added therein to dissolve at 50° C. to 60° C. as a saturated solution with pH 1.2. The saturated solution is sequentially decolorized with activated charcoal, filtered, deposited overnight and filtered to obtain a white crystal, i.e. Pulmodil. Pulmodil has a chemical formula as 7-[2-[4-(2-chlorobenzene)piperazinyl]ethyl]-1,3-dimethylxanthine. HCl, which has a melting point of 249° C. to 252° C. The reaction formula is illustrated as follows.

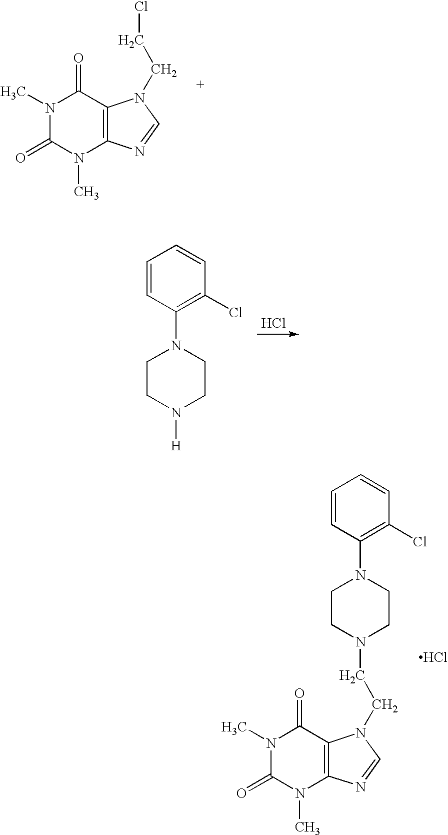

### Embodiment 2

### Synthesis of Pulmodil-1

The second preferred embodiment of the present invention is Pulmodul-1. Method 1: 2-Chloroethyl theophylline, 2-chlorophenyl piperazine and NaOH (or NaHCO₃) are dissolved in hydrous ethanol solution based on the molecular weight percentage and heated under reflux for three hours. After cooled overnight, the supernatant is decanted for proceeding the vacuum concentration and dry process, and then, ethanol and citric acid at a ratio of 1:1 (mole/mole) are added therein to dissolve at 50° C. to 60° C. as a saturated solution with pH 4.0. The saturated solution is sequentially decolorized with activated charcoal, filtered and deposited overnight to obtain a white crystal, i.e. Pulmodil-1. Pulmodil-1 has a chemical formula as 7-[2-[4-(2-chlorobenzene)-piperazinyl]ethyl]-1,3-dimethylxanthine.citric acid, and the reaction formula is illustrated as follows.

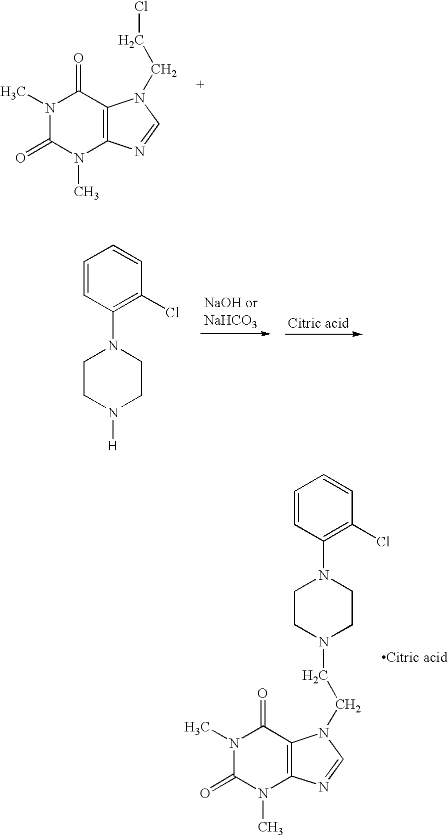

Method 2: 2-Chloroethyl theophylline, 2-chlorophenyl piperazine are dissolved in hydrous ethanol solution based on the molecular weight percentage and heated under reflux for three hours. After cooled overnight, the supernatant is decanted for proceeding the vacuum concentration and dry process, and then, ethanol and citric acid at a ratio of 1:1 (mole/mole) are added therein to dissolve at 50° C. to 60° C. as a saturated solution with pH 4.0. The saturated solution is sequentially decolorized with activated charcoal, filtered and deposited overnight to obtain a white crystal, i.e. Pulmodil-1. Pulmodil-1 has a chemical formula as 7-[2-[4-(2-chlorobenzene)piperazinyl]ethyl]-1,3-dimethylxanthine.citric acid, and the reaction formula is illustrated as follows.

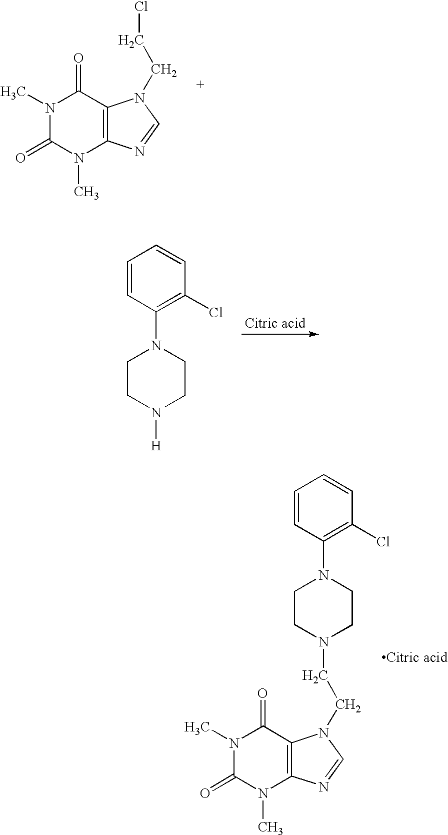

The above mentioned two chlorophenylpiperazine salt derivatives, Pulmodil and Pulmodil-1, are merely preferred embodiments of the present invention, wherein the hydrochloride of Pulmodil could be substituted by other inorganic acid, such as a sulfuric acid, a phosphoric acid, a boric acid or a dihydrochloride, and the citric acid of Pulmodil-1 could be substituted by other organic acid, such as a maleinic acid, a fumaric acid, a tartaric acid, an oleic acid, a stearic acid, a benzenesulphonic acid, an ethyl benzenesulphonic acid, a benzoic acid, a succinic acid, a mesylic acid, a dimesylic acid, an acetic acid, a propionic acid, a pentanoic acid or an aspartic acid. During the synthetic process, various inorganic acid or organic acid could be used to produce different chlorophenylpiperazine salt derivatives. For example, when the inorganic acid is sulfuric acid, the derivative, 7-[2-[4-(2-chlorobenzene)-piperazinyl]ethyl]-1,3-dimethylxanthine.Sulfuric acid, could be obtained; and when the organic acid is maleinic acid, the derivative, 7-[2-[4-(2-chlorobenzene)-piperazinyl]ethyl]-1,3-dimethylxanthine.Maleinic acid, could be obtained. The rest may be deduced by analogy.

Regarding the chemical structure, Pulmodil has a main structure and an HCl molecule, and Pulmodil-1 has the same main structure and a citric acid. Although the main structure of Pulmodil can be obtained by the reaction of 7-ethylbromotheophylline with 1-(2-chlorophenyl)-piperazine, Pulmodil can be obtained by the synthetic method disclosed in the present invention without the preparing steps, such as filtration and recrystallization, etc., of the main structure compound (hereinafter referred to as “MSC”). It is apparent that the method disclosed in the present invention directly completes the synthesis of Pulmodil or Pulmodil-1 without a prior step of synthesizing a compound with the main structure. Further, since one reactant of the abovementioned reaction is 1-(2-chlorophenyl)-piperazine, instead of 1-(2-bromophenyl)-piperazine, the method disclosed in the present invention will not have a risk involving the contamination of bromine. In addition to the different synthetic methods between Pulmodil/Pulmodil-1 and the MSC, the physiochemical and physiological differences therebetween are described in detail as follows.

(1) Melting Point:

The melting point of MSC is ranged between 168° C. and 172° C., which is significantly lower than the melting point of Pulmodil (249° C. to 252° C.).

(2) HPLC Analysis:

For proving that Pulmodil is a single molecule but not dissociated into two parts, MSC and HCl, which perform functions, respectively, when functioning in on organism, purity of Pulmodil is determined by HPLC. First, Pulmodil and the standard solutions with different concentrations are prepared, wherein the standard is a compound having a chemical formula, C₁₅H₁₄ClNO₂S.HCl, with the molecular weight of 344.26. Pulmodil is dissolved in a solution containing 25% acetonitrile and 0.1% formic acid. After a serial dilution, a series concentrations (0, 0.1, 0.2, 0.5, 1, 2, 5, 10 and 20 ng/ml) of the standard solutions are used in HPLC to calculate the low-concentration standard curve, and a series concentrations (0, 10, 20, 50, 100, 200, 500, 1000, 2000 and 5000 ng/ml) thereof are used in HPLC to calculate the high-concentration standard curve. The standards are dissolved in a solution containing 50% acetonitrile, and then diluted with 100% acetonitrile. The concentration of the diluted Pulmodil is 18 ng/ml in the low-concentration standard curve and 450 ng/ml in the high-concentration standard curve, respectively. The column for HPLC is Luna C18 column (2.0 mm×50 mm, 5 mm, Phenomenex), in which the mobile phase includes 23% acetonitrile and 1.0% formic acid, and the flow rate is about 0.2 ml/min.

The parameters of tandem mass spectrometry are set as follows: capillary: 3.2 kV, cone: 40 V; source temperature: 80° C.; desolvation temperature: 400° C.; collision of 20 V; and multiplier: 500 V.

Pulmodil represents parent ion of 403.12 m/z (mass-to-charge ratio) and daughter ion of 222.95 m/z by the analysis of the tandem mass spectrometry, and standard represents parent ion of 308 m/z and daughter ion of 197.96 m/z thereby.

Please refer to Tables 1 and 2, which respectively represent the analytic results of Pulmodil solutions with low- and high-concentrations in HPLC. The result of peak-covered area is calculated according to the retention time of the sample, and the area percentage represents the purity of the material in the sample. It could be known from Tables 1 and 2 that Pulmodil has a purity of 100% in the samples. It shows that Pulmodil is still a single compound after being dissolved.

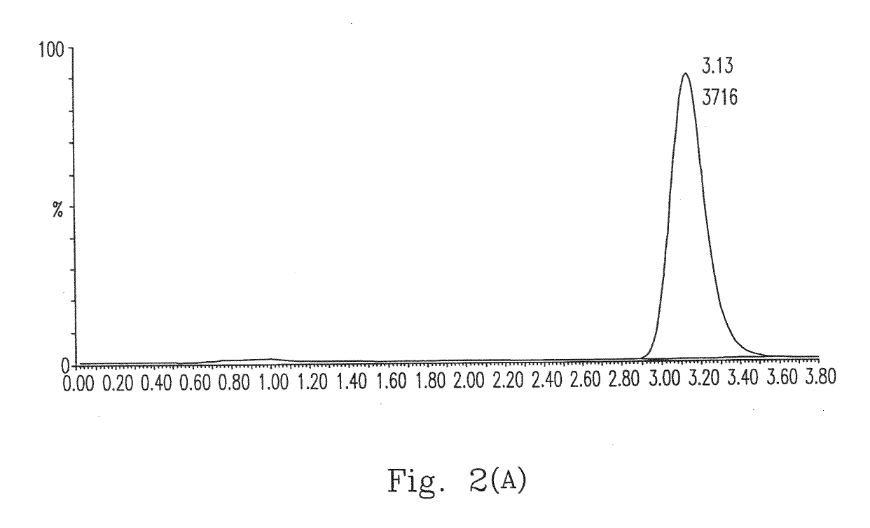

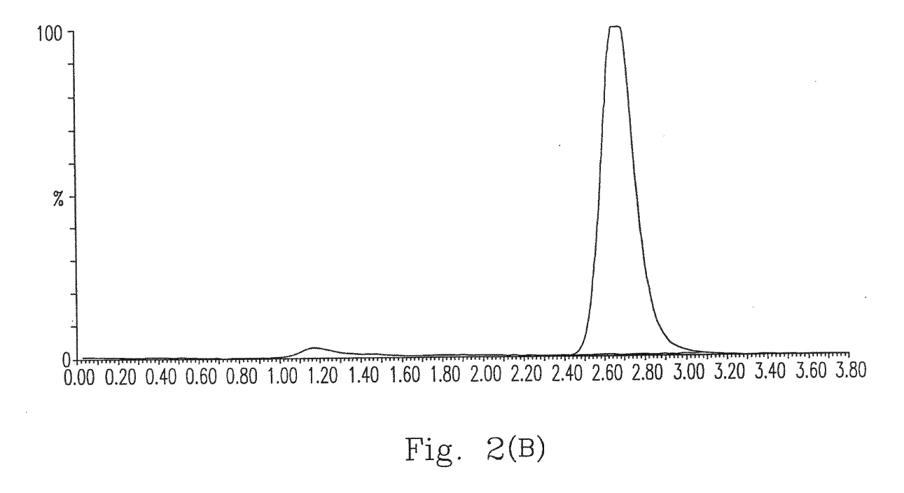

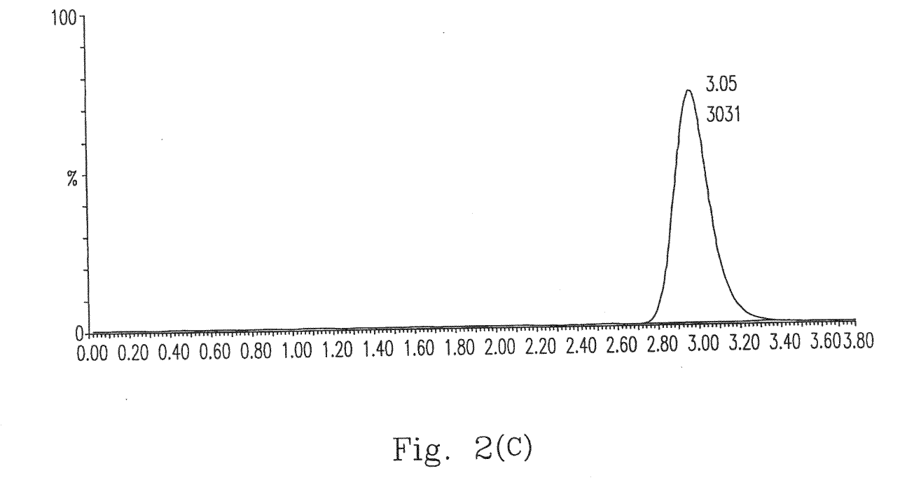

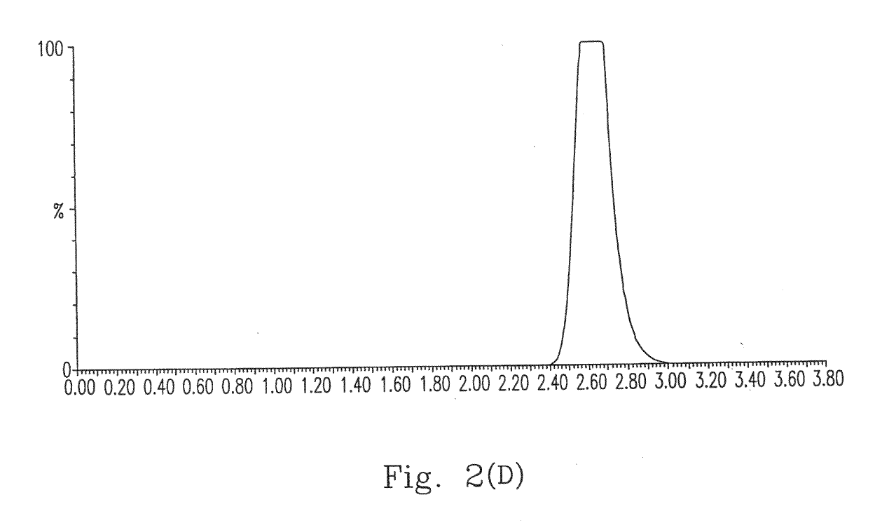

Please refer to FIGS. 2 (A) to (D), which respectively show the comparison results between 5 ng/ml Pulmodil (A) and the standard (B) and between 200 ng/ml Pulmodil (C) and the standard (D). In FIGS. 2 (A) and (C), Pulmodil represents a peak at about 3 minutes, and no other minor peak is shown. It means that no other molecule exists in the solution.

TABLE 1

| Concentration | Retension time | Area (uv × | Area | |
|---|---|---|---|---|
| (ng/ml) | (min) | sec) | % | Height |
| 0.1 | 3.10 | 71.10 | 100 | 382 |
| 0.2 | 3.12 | 145.67 | 100 | 710 |
| 0.5 | 3.06 | 359.96 | 100 | 1759 |
| 1 | 3.09 | 801.44 | 100 | 3914 |
| 2 | 3.10 | 1463.25 | 100 | 7111 |
| 5 | 3.13 | 3716.39 | 100 | 17876 |
| 10 | 3.08 | 6919.70 | 100 | 34354 |
| 20 | 3.06 | 14481.06 | 100 | 71196 |

TABLE 2

| Concentration | Retension time | Area (uv × | Area | |
|---|---|---|---|---|
| (ng/ml) | (min) | sec) | % | Height |
| 10 | 3.08 | 143.95 | 100 | 706 |
| 20 | 3.04 | 320.91 | 100 | 1580 |
| 50 | 3.01 | 800.71 | 100 | 3955 |
| 100 | 3.01 | 1494.92 | 100 | 7303 |
| 200 | 3.05 | 3030.55 | 100 | 14663 |
| 500 | 3.05 | 7807.26 | 100 | 37646 |
| 1000 | 3.05 | 14499.72 | 100 | 70052 |
| 2000 | 3.01 | 28783.05 | 100 | 140845 |
| 5000 | 3.01 | 77175.41 | 100 | 368520 |

(3) Solubility Test:

Solubilities of three compounds, i.e. Pulmodil, Pulmodil-1 and MSC, disclosed in the present invention are evaluated and the results are shown in Table 3. It is found that Pulmodil-1 and MSC are necessary to be dissolved by using a surfactant. As shown in Table 3, MSC can be dissolved in a solution containing propylene glycol and Pulmodil-1 can be dissolved in the solution containing polyethylene glycol (PEG). However, a drug prepared from a compound with too much organic solvent is dangerous. Pulmodil-1 and MSC solutions are acidic and not adequate for the general physiological environment. Comparing with Pulmodil-1 and MSC, Pulmodil could be dissolved in 5% (w/v) glucose solution, has better solubility and is easily prepared. Pulmodil is safe in usage since glucose solution is a popular medical supplement. Furthermore, the pH value of the dissolved Pulmodil is about 5.8 to 6.4, which closes to the general physiological environment.

TABLE 3

| | Concentration | | |
|---|---|---|---|
| Compound | (mg/ml) | Solvent | pH |
| MSC | 100 | Anhydrous EtOH:PEG | 4.2-4.8 |
| | | 400:1N HCl:H₂O = | |
| | | 10:10:3:77 (v/v) | |
| | 90 | 1.2 ml Propylene glycol | 4.2-4.8 |
| | | and 0.6 ml 1N HCl | |
| Pulmodil | 8 | 5% Glucose | 5.8-6.4 |
| Pulmodil-1 | 20 | Anhydrous EtOH:PEG | 4.5-4.8 |
| | | 400:H₂O = 5:30:65 (v/v) | |

In pharmaceutics, the water-soluble Pulmodil disclosed in the present application is facile to be formulated. Furthermore, Pulmodil has the biological effects on inhibiting a pulmonary artery endothelium dysfunction, a thickened pulmonary artery medial wall, and a vascular obstruction, which are proved by the following experimental results.

Hereinafter are the detailed descriptions of the biological activity tests of Pulmodil.

### Embodiment 3

### Normoxia Model

1. Incubation of the Tracheal Smooth Muscle Cells (TSMCs):

The tracheal tissue of Wistar rat (200 g to 250 g) is aseptically obtained and the connective tissue around the tracheal tissues is removed. After clearance, tracheal tissue is aseptically sliced as fragments and spread on the T-25 flask. The T-25 flask is added with 6 ml DMEM medium (containing 20% (v/v) feotal bovine serum (FBS)) and incubated in a 37° C. incubator with 5% CO₂. Later, the medium is refreshed by medium B (DMEM supplemented with 10% FBS) per three days. When 80% to 90% cell confluence was achieved, subcultures are performed.

The subculture process includes the following steps: decanting the medium at 80% to 90% cell confluence, rinsing cells with 2 ml phosphate buffered saline (PBS) once or twice, adding 1 ml solution including 0.25% trypsin and 0.02% EDTA (ethylenediaminetetraacetic acid) and incubated under 37° C., adding 10 ml medium B to cease the function of trypsin when cells come off the surface of the flask, collecting the medium containing the cells in the sterile centrifuge tube and discarding the supernatant after the centrifugation, resuspending cells with 10 ml fresh medium B, and culturing some suspended cells on 10 cm cell culture plate to proceed the subsequent experiments. Third to sixth generations of cells are used in the following experiments.

2. Identification of TSMCs:

First, morphology of the smooth muscle cells is observed under an optical microscope, and then the immunofluorescent assay described below is used to determine whether cells have a-actin.

The sterile cover glasses are disposed into the wells of 24-well culture plate and rat TSMCs are added into the wells by the concentration of 5×10³ cells/ml per well. The cells are incubated at 37° C. in a humidified atmosphere of 5% CO₂/95% O₂ overnight to make cells attach on the surface of the cover glasses. The medium in the wells is removed and cells are washed with 0.5 ml ice-cold PBS solution for triple times. 1 ml formaldehyde is added into each well for 5 minutes at room temperature to fix cells. After removing the formaldehyde, PBS solution (1 ml) is added into each well and the culture plate is gently shaken on horizontal orbital shaker for 5 minutes. After removing the PBS solution, 0.5 ml prepermeabilized lysis buffer (BD. Pharmingen, San Diego, Calif.) is added into each well and reacted with cells for 15 minutes at room temperature. After removing the prepermeabilized lysis buffer, the cells are washed with PBS solution for triple times, 0.5 ml for each time. 0.5 ml FITC-conjugated monoclonal mouse anti-smooth muscle a-actin antibody (1:100 dilution) is added into the culture plate to darkly react for 2 hours at room temperature. After the reaction of the antibody, the wells are washed with washing buffer (containing 20 mM Tris base, 140 mM NaCl, 1% (v/v) Tween 20, pH 7.6) twice, and then the cover glasses are mounted on the glass slide with the fluorescent mounting medium for 30 minutes and visualized using a fluorescent microscope.

Please refer to FIGS. 3 (A) and (B), which respectively show the primary cultured TSMCs under (A) phase contrast microscope and (B) fluorescence microscope. The field in FIG. 3 (B) is identical to that in FIG. 3 (A). The smooth muscle cells are marked green by the antibody. It could be known from the comparison between FIGS. 3 (A) and (B) that almost all the cells represent green fluorescence under the fluorescence microscope, which means that the primary cells are almost all smooth muscle cells.

3. MTT Assay:

Cell concentration is adjusted to 10⁴ cells/ml by using a cell counter. Ten thousand cells (1 ml) are inoculated in 24-well culture plate for 24 hours, and cells are attached thereon. Medium B then is added into the culture plate and cells are cultured for another 24 hours. Subsequently, different concentrations of drugs are treated for 24 hours in low-oxygen incubator. 100 μL MTT (methylhiazolyldiphenyl-tetrazolium bromide, 5 mg/ml) is added darkly into the wells and the reaction is proceeded darkly at 37° C. for 3 hours. After MTT is decanted, 500 μL Isopropanol is added into the wells and the culture plate is shaken for 10 minutes and incubated for another 10 minutes. After the incubation, the supernatant (200 μL) is transferred to a new 96-well culture plate for determining the absorbance at 540 nm (OD₅₄₀) and 630 nm (OD₆₃₀). The effect of drugs on cell growth is evaluated by the value of “OD₅₄₀-OD₆₃₀”.

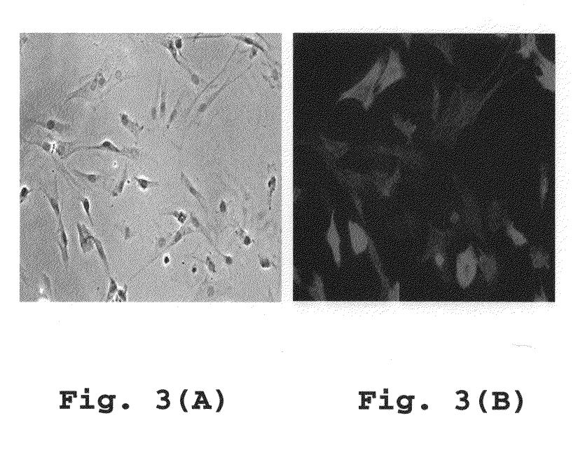

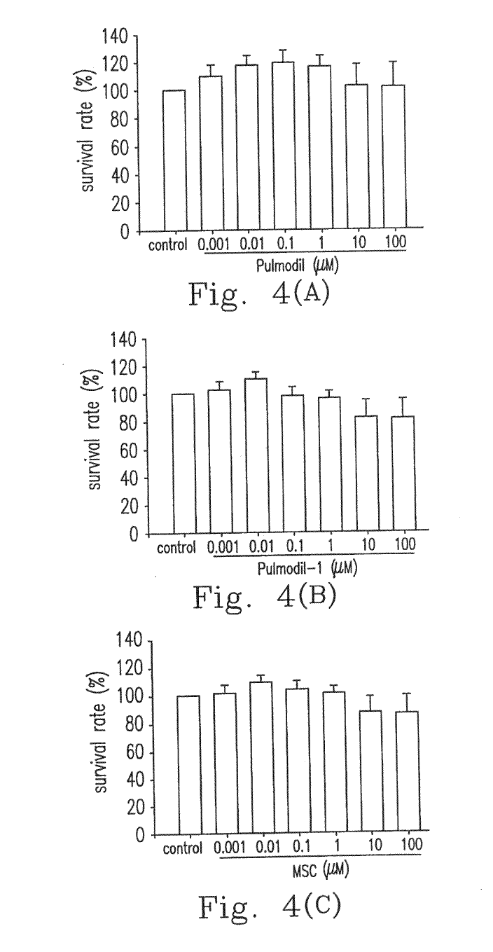

Please refer to FIGS. 4 (A) to (C), which respectively show the effects of different concentrations of Pulmodil, Pulmodil-1 and the major structural compound (MSC) on the survival rates of the TSMCs of Wistar rat. As shown in FIGS. 4 (A) to 4 (C), it could be known from the MTT assay that different concentrations (0.001 to 100 μM) of Pulmodil show no cytotoxicity on rat TSMCs; in particular, the cell survival rate almost achieves 100% at high concentrations (10 and 100 μM). Comparing with Pulmodil, Pulmodil-1 and MSC still represent little toxicity to cells, and the cell survival rate is about 80% to 85% at high concentrations (10 and 100 μM).

Usage of Pulmodil is much safer than that of Pulmodul-1 or that of MSC in accordance with the results of MTT assay. Particularly, Pulmodil is a salt derivative of its major structural compound, and the structural difference therebetween is a hydrochloride molecule. However, Pulmodil represents a significant improvement in toxicity and gains the great benefits on medical treatment.

4. The Effect of Pulmodil on U46619 Induced Pulmonary Artery Hypertension:

Acute thromboxane A2 (TXA2)-mimetic U46619 can cause the hypertension and reduce oxygen content of the pulmonary artery by invoking the contraction of the pulmonary artery, and U46619 also results in the lack of NO by inactivating the function of eNOS and thus results in the decreased expression of cGMP/PKG. Therefore, the effects of Pulmodil on the mechanism of U46619 are studied by the pretreatment of U46619.

Hemodynamic measurement of mean arterial pressure (MAP) is carried out in male Wistar rats, weighting 300˜0350 g, anesthetized with pentobarbital sodium (40 mg kg⁻¹) by intraperitoneal injection (hereinafter referred to as “i.p.”).A catheter is inserted into femoral artery, and the MAP and heart rate are recorded with a pressure transducer (Gould, Model P50, U.S.A.) connected with the catheter and a Pressure Processor Amplifier (Gould, Model 13-4615-52, U.S.A.). The catheter in the femoral vein could be used for the intravenous administration of Pulmodil and other drugs.

The mean pulmonary arterial pressure (MPAP) is recorded by catheterizing the pulmonary artery in closed-chest rats with a PE-50 catheter connected with a disposable diaphragm dome TA1019, a pressure transducer and an amplifier.

After hemodynamic measurement is balanced, Pulmodil (0.5-2.0 μg kg⁻¹ min⁻¹) and reference drugs, such as milrinone (1 μg kg⁻¹ min⁻¹), sildenafil (1 μg kg⁻¹ min⁻¹), zaprinast (10 μg kg⁻¹ min⁻¹), urapidil (100 μg kg⁻¹ min⁻¹), are administrated by intravenous infusion (hereinafter referred to as “i.v.”) for 20 minutes and subsequently followed by continuous intravenous infusion of U46619 (2.5 μg kg⁻¹ min⁻¹) for 30 minutes to achieve approximately a 2-fold elevation in MPAP of U46619 as control level. If Pulmodil is administrated by intraperitoneal injection, the dosage could be about 0.1-1.0 mg kg⁻¹.

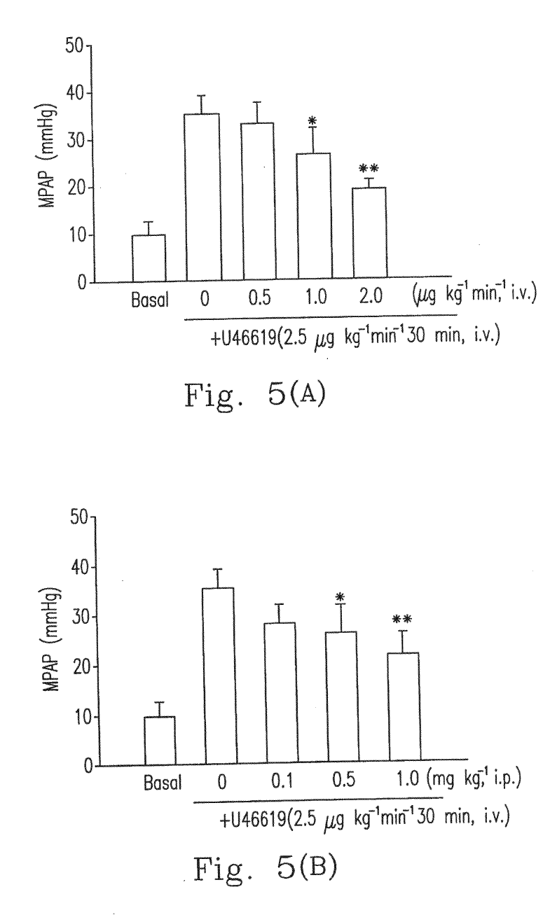

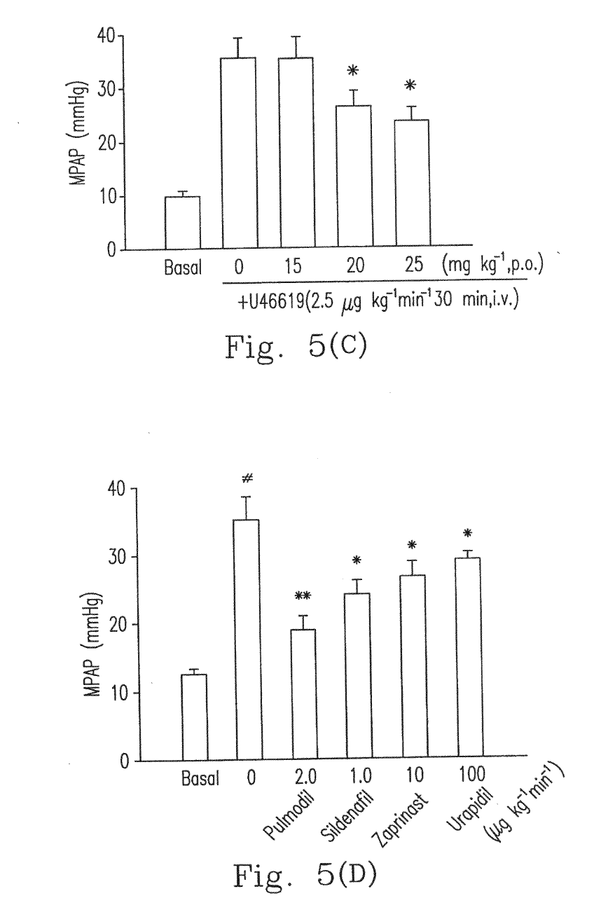

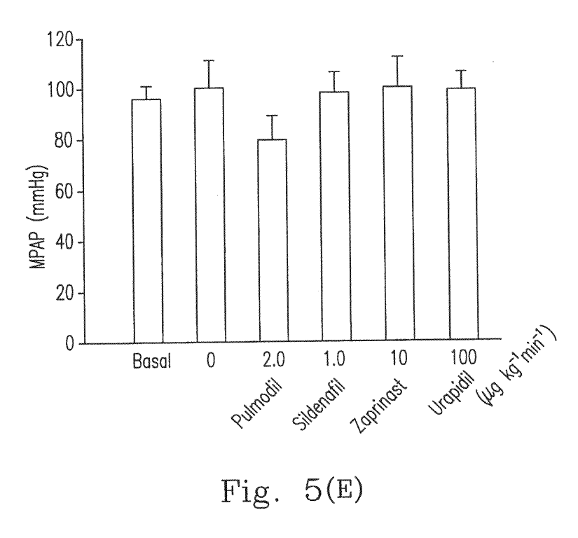

Please refer to FIGS. 5 (A) to (C), which show the effects of Pulmodil with different concentrations on the mean pulmonary arterial pressure (MPAP) in U46619-treated rats. As shown in FIGS. 5 (A) to (C), intravenous infusion of U46619 for 30 min produces a significant increase of rat MPAP, and different routes of pretreatment with Pulmodil can prevent from subsequent U46619-induced PAH. As shown in FIG. 5 (A), intravenous Pulmodil (0.5˜2.0 μg kg⁻¹ min⁻¹, i.v., 20 min) can significantly decrease the MPAP caused by U46619 from 35.2±3.9 to 33.0±4.5, 26.4±5.6 and 18.8±2.1 mmHg, respectively. Please refer to FIG. 5 (B), intraperitoneal Pulmodil (0.1˜1.0 mg kg⁻¹, i.p.) can significantly decrease the MPAP caused by U46619 from 35.2±3.9 to 28.0±3.8, 26.0±5.6 and 21.5±4.5 mmHg respectively. Please refer to FIG. 5 (C), oral administration (i.e. per os, p.o.) of Pulmodil (15-25 mg kg⁻¹, p.o.) also prevents from subsequent U46619 infusion-induced increase of MPAP to 35.4±4.0, 26.3±2.9 and 23.5±2.5 mmHg without significant effects on mean artery pressure.

Please refer to FIGS. 5 (D) and (E), which show the effects of various drugs on the mean pulmonary arterial pressure (MPAP) in U46619-treated rats. As shown in FIGS. 5 (D) and 5 (E), at tolerable maximal dose, without significant systemic effect on MABP, milrinone (1 μg kg⁻¹ min⁻¹, 20 min), sildenafil (1 μg kg⁻¹ min⁻¹, 20 min) and zaprinast (10 μg kg⁻¹ min⁻¹, 20 min) all decreased MPAP, but urapidil is effective to decrease MPAP at higher dose (100 μg kg⁻¹ min⁻¹, 20 min). Based on the above, it could be known the sequence of potency is Pulmodil, sildenafil, PDE 5 inhibitor zaprinast and a-adrenoceptor antagonist urapidil from strong to weak. Pulmodil decreased MABP at maximally effective dose (2 μg kg⁻¹ min⁻¹, 20 min) during inhibiting MPAP, without sudden death of rats after subsequent perfusion with U46619, while Pulmodil at this higher dose mildly decreased mean arterial blood pressure (MABP) to 17% .

5. The Effect of Pulmodil on the Monocrotaline (MCT) Induced Chronic Pulmonary Artery Lesions:

Eight-week-old adult Wistar rats are given with a single injection of monocrotaline (MCT, 60 mg kg⁻¹, i.p.) on day 0 for inducing chronic PAH. Pulmodil (5 mg kg⁻¹ day⁻¹, p.o. and 1 mg kg⁻¹ day⁻¹, i.p. for 21 days) is administered to prevent from worsening of PAH. As shown in Table 4, after the treatment of Pulmodil for 21 days, MCT-induced chronic PAH is acutely decreased.

TABLE 4

| | MABP | Heart Rate | MPAP |
|---|---|---|---|
| | (mmHg) | (beats/min) | (mmHg) |
| Control | 114.5 ± 6.4 | 365.2 ± 20.0 | 12.3 ± 1.2 |
| MCT | 102.5 ± 8.2 | 344.0 ± 18.1 | 30.7 ± 1.2^{##} |
| Pulmodil (5 mg, p.o.) + | 104.4 ± 5.4 | 342.5 ± 11.7 | 21.8 ± 2.9* |
| MCT | | | |
| Pulmodil (1 mg, i.p.) + | 100.2 ± 4.0 | 384.4 ± 17.1 | 18.7 ± 4.6* |
| MCT | | | |

6. Pulmonary Artery Relaxation Tension Measurement:

The isolated rat pulmonary artery rings (2˜3 mm) are suspended under isometric conditions and connected to a force transducer (UGO BASLINE, Model 7004, Italy). Cumulative concentration-response curves are constructed in response to TXA2 receptor agonist U46619 (0.5 μM) and a-adrenoceptor agonist phenylephrine (PE, 10 μM). Amplitude of the contraction is expressed as a percentage of the maximal U46619-induced and PE-induced contraction. The amplitude of relaxation is expressed as percentage of the maximal amplitude of contraction induced by U46619 or PE application.

After equilibration, rings are contracted with U46619 or PE. When the contractile response to each agonist reaches a stable tension, cumulative concentration-response curves to Pulmodil (0.01˜100 μM) are carried out by cumulative addition of them after a steady-state response is reached after each increment. To examine the possible mechanisms of pulmonary artery relaxant effects of Pulmodil in some experiments, the relaxant effects of Pulmodil is tested in pulmonary arteries pretreated with sGC inhibitor ODQ (1 μM), NOS inhibitor L-NAME (100 μM) and adenylate cyclase inhibitor SQ22536 (1.0 μM), before a contraction was induced with U46619 (0.5 μM) or PE (1.0 μM). Contractile tension is recorded by a computer program. The preparations are stretched to a resting tension of 1 g, and allowed to equilibrate for 60±90 min.

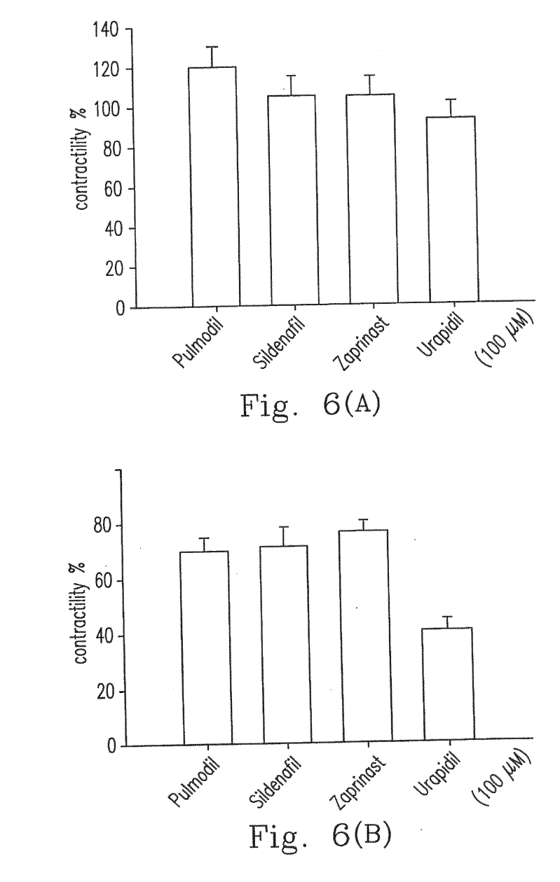

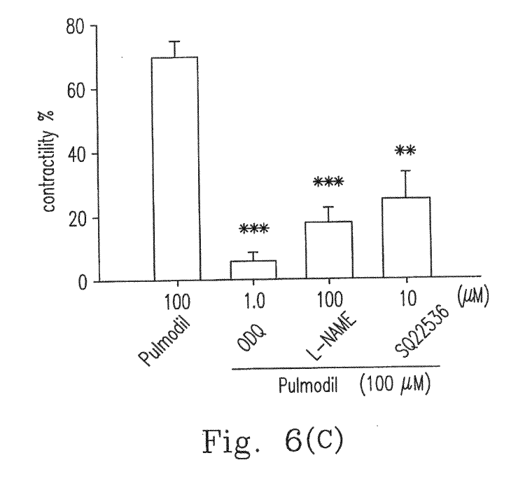

Please refer to FIGS. 6 (A) to (C), which showing the effects of Pulmodil (100 μM) on phenylephrine and U46619 induced contractility of the pulmonary artery rings. As shown in FIGS. 6 (A) and (B), in the isolated rat pulmonary arterial rings, Pulmodil (100 μM) relaxed phenylephrine- (FIGS. 6 (A)) and U46619- (FIG. 6(B)) induced contractility to 120±10.2% and 69.8±4.9%, respectively. Milrinone, zaprinast and urapidil at 100 μM also relax penylephrine- and U46619-induced contractions. Among the abovementioned drugs, a-adrenoxeptor antagonist urapidil is the weakest one to relax U46619-induced vasocontraction (shown in FIG. 6 (B)), which indicates that Pulmodil might be less unrelated to the blockade of the a-adrenoceptor since Pulmodil markedly inhibits U46619-induced contractions. However, such relaxation by Pulmodil is blunted by the pretreatments with a NOS inhibitor L-NAME and a sGC inhibitor ODQ from 69.8±4.9% to 17.7±4.6% and 5.8±2.7% respectively, indicating the predominate cGMP-dependency of Pulmodil (as shown in FIG. 6(C)). The relaxation caused by Pulmodil is also reduced by the pretreatment with adenylyl cyclase inhibitor SQ22536 from 69.8±4.9% to 24.9±8.5%, demonstrating the less involvement of cAMP than cGMP in Pulmodil.

In Animal models of either acute or chronic PAH, Pulmodil causes a significant decrease of pulmonary artery pressure, without affecting systemic artery pressure. Further, based on the experiments regarding the pulmonary artery vasodilatation, it could be known that Pulmodil relieves the pulmonary artery hypertension by relaxing the contractions of the pulmonary artery rings.

In addition, it is known hypertension is also a risk factor for arteriosclerosis. When Pulmodil of the present invention is used to relieve the pulmonary artery hypertension, it is found that the risk of suffering a cardiovascular disease including a angina, a myocardial Infarction and a heart failure, a cerebral vascular accident, a diabetes, a diabetic retinopathy or a nephropathy is also decreased.

7. Blood Oxygenation Assay:

Blood samples are simultaneously obtained (at pretreatment and the end of each treatment) from femoral arteries, following intra-peritoneal injection and intravenous infusion of Pulmodil and reference agents. After final administration, blood is sampled under conscious conditions through the catheter and subjected to analysis using an automatic blood gas analyzer (AVL OMN™, U.S.A.). The analysis included arterial oxygen pressure (PaO₂), arterial carbon dioxide pressure (PaCO2), pH value, O₂ saturation (SO₂%). U46619 (2.5 mg kg⁻¹ min⁻¹) alone decreases PaO₂ to 89±3.1 mmHg in plasma of Wistar rats. Pretreatment with Pulmodil by intravenous infusion reverses this condition to 100.1±2.2 mmHg at the dose of 1 μg kg⁻¹ min⁻¹. U46619-induced decreases of plasma oxygenation is restored by sildenafil (1 μg kg⁻¹ min⁻¹, i.v.) to 100.6±3.0, and zaprinast (10 μg kg⁻¹ min⁻¹, i.v.) to 98±3.1 (as shown in Table 5).

TABLE 5

| | | | Pulmodil | Sildenafil | Zaprinast | Urapidil |
|---|---|---|---|---|---|---|
| | Control | U46619 | +U46619 | | | |
| PaO₂ (mmHg) | 103 ± 10 | 89 ± 3.1 | 100.1 ± 2.2 | 100.6 ± 3.0 | 98 ± 3.1 | 91.5 ± 3.1 |

8. The effects of Pulmodil on Ca²⁺-activated K⁺ (BK_{Ca}) currents:

Smooth muscle cells from rat pulmonary arteries are enzymatically isolated. After this equilibration step, arterial segments are incubated (37° C.) in 0.5 mg/ml collagenase IA, 0.6 mg/ml papain and 0.2 mg/ml dithioerythritol for 45 min. After enzyme treatment, the tissues are washed three times in ice cold isolation medium and triturated with a fire polished pipette to release the myocytes. Cells are stored in ice-cold isolation medium for use in the same day. Whole cell BK_{Ca} currents are measured using the conventional patch-clamp configuration.

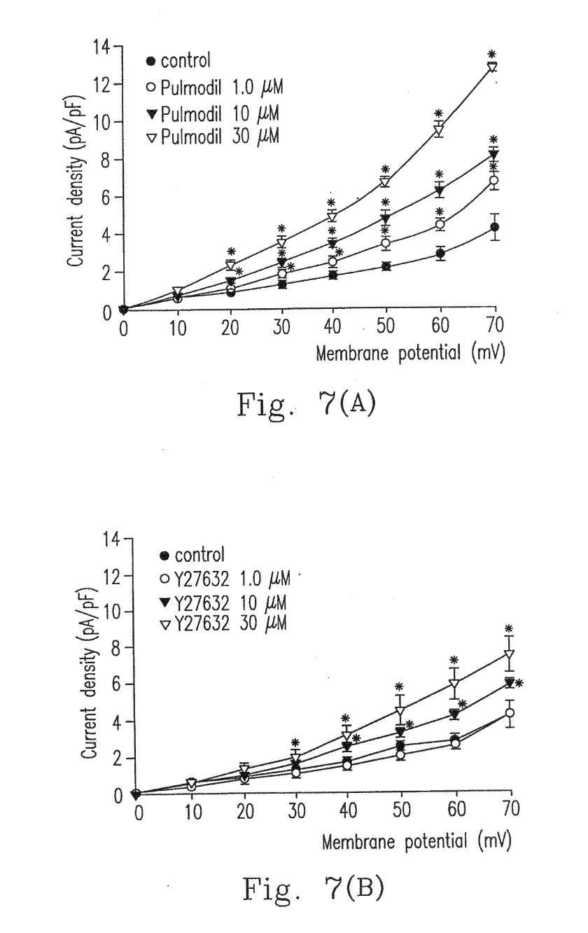

Please refer to FIGS. 7(A) and (B), which shows the effects of Pulmodil on K⁺-channel opening activity in the pulmonary artery smooth muscle cells. In pulmonary artery smooth muscle cells of normal rats, Pulmodil (1˜30 μM), more potently than Y27632 (1˜30 μM), opens K⁺-channel in cell membrane.

9. The Effects of Pulmodil on Ca²⁺ Mobilization and Concentrations:

The measurements of Ca²⁺-mobilization and concentrations of rat epithelial cell line are performed by a spectrofluorophotometer. Both cultured rat epithelial cell and primary pulmonary artery smooth muscle cells are loaded with Fura-2/AM to permit the measurements of Ca²⁺-concentration changes in single cells by a spectrofluorophotometer (Shimadzu, RF-5301PC, Japan).

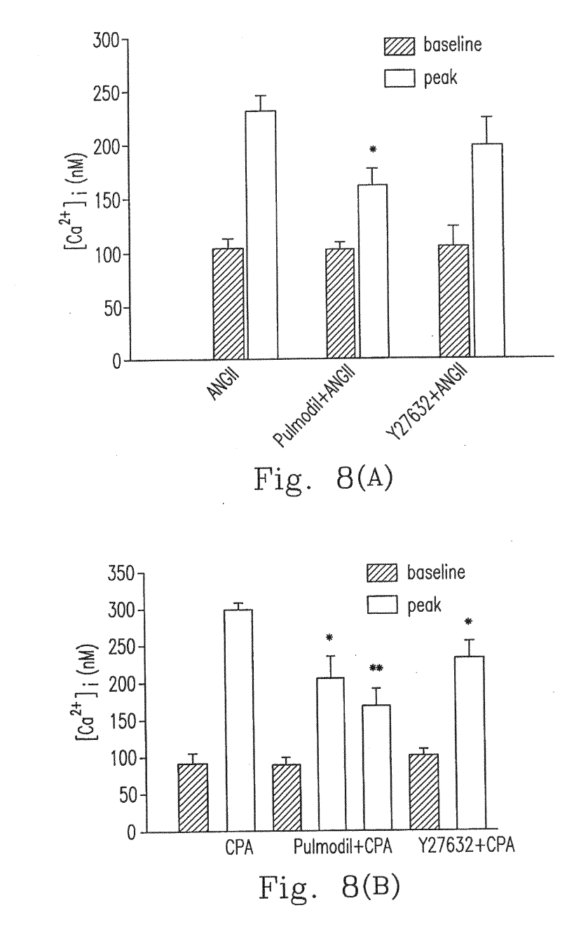

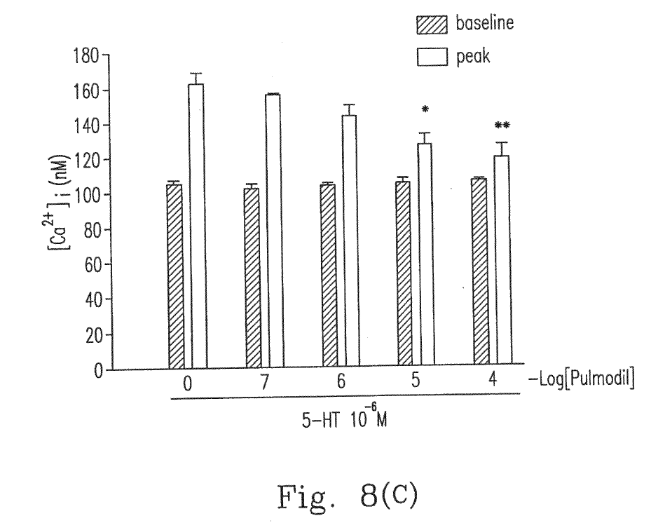

Please refer to FIGS. 8 (A) to (C), which shows the effects of Pulmodil and Y27632 on different drugs-induced Ca²⁺ mobilization. Pulmodil is more potent than Y27632 to inhibit angiotensin II (ANGII), cyclopiazonic acid (CPA) and 5-HT-induced influx of Ca²⁺ in calcium-containing buffer.

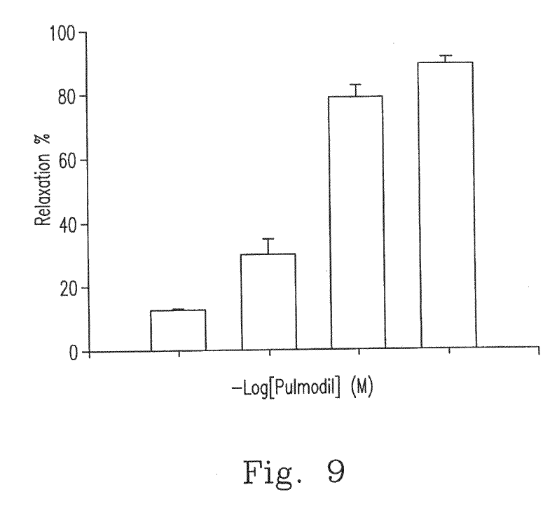

Please refer to FIG. 9, which indicates that Pulmodil inhibits not only the 5-HT-induced Ca²⁺-influx, but also the smooth muscle contractions.

K⁺ and Ca²⁺-mobilization/sensitization regulate pulmonary vascular tension and remodeling and constitute potential therapeutic targets in the regression of PAH. Loss of K⁺-channel and opening activity may contribute to the pathogenesis of PAH by causing a sustained depolarization, which increases intracellular Ca²⁺ and K⁺, thereby stimulating cell proliferation. Therefore, it could be known a medial thickness and an endothelium dysfunction of pulmonary artery, and a vascular obstruction could be caused by the reduced K⁺-channel and the lost opening activity thereof.

10. The Effects of Pulmodil on the Expressions of eNOS/sGC/PKG/PDE5A/ROCKII Proteins:

Isolated pulmonary arterial rings are incubated in cell culture medium in the presence of U46619 (0.5 μM) for 60 min and then treated with Pulmodil (0.1˜100 μM) for 60 min. Expressions of the relevant proteins are analyzed using their mouse or rabbit monoclonal antibodies, respectively. Immunoreactive bands are visualized using horseradish peroxidase-conjugated secondary antibodies and subsequent ECL detection (Amersham Pharmacia, USA). Pretreatment with various inhibitors for 30 min before application of Pulmodil or the reference agents is followed by the same procedure.

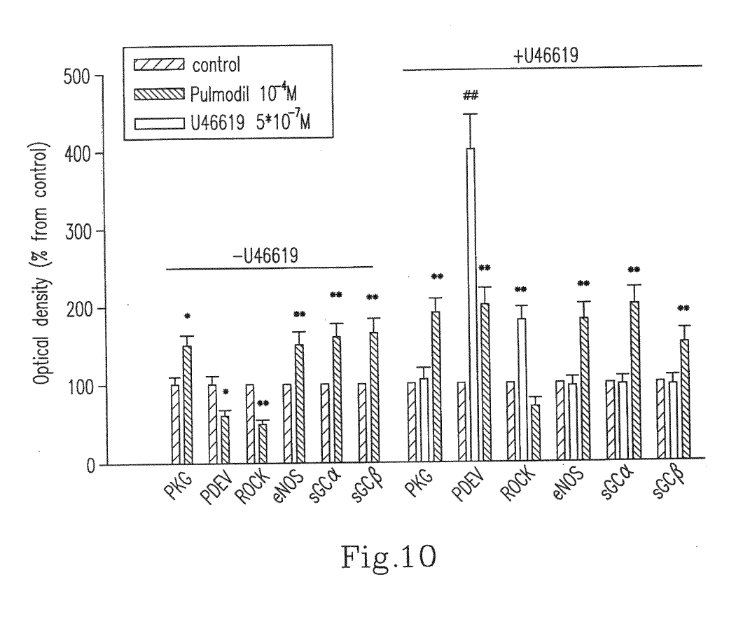

Please refer to FIG. 10, which shows the effects of Pulmodil on the expressions of eNOS/sGC/PKG/PDE5A/ROCKII proteins in the U46619-treated rats. In the presence or absence of U46619 (0.5 μM), Pulmodil (100 μM) increases eNOS, sGC and PKG and particularly decreases PDE5A and ROCKII expressions in intact isolated pulmonary artery. Since Pulmodil of the present invention could activate cGMP and inhibit U46619-induced PDE5A, it could indeed inhibit a medial thickness and an endothelium dysfunction of pulmonary artery, and a vascular obstruction through the combination of the abovementioned two activities.

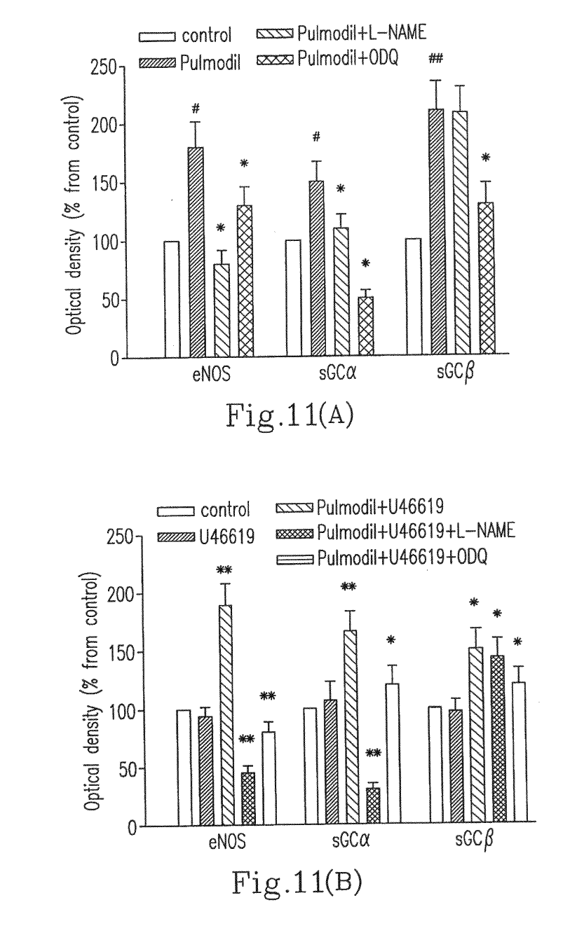

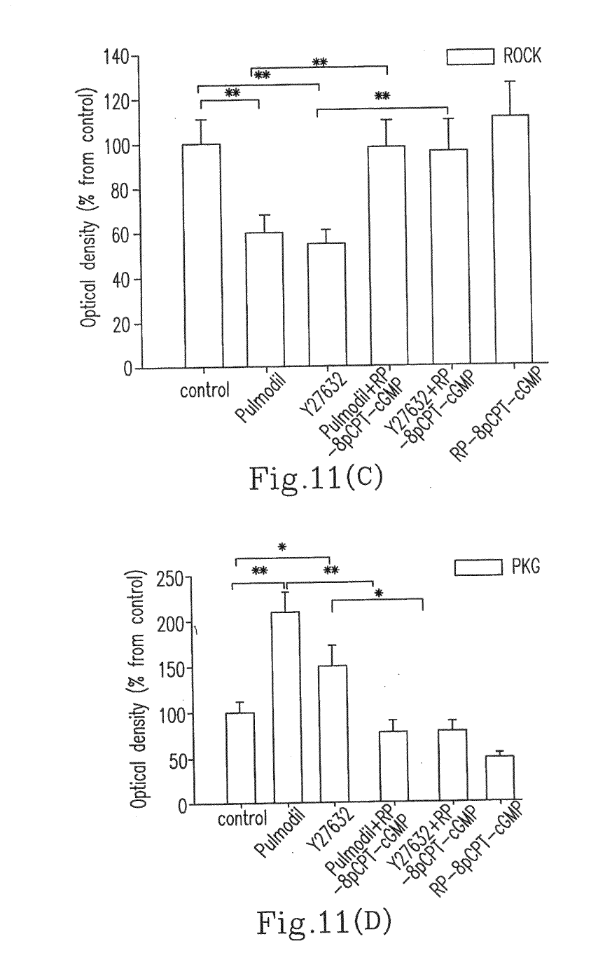

Please refer to FIGS. 11 (A) to (D), which shows the effects of Pulmodil on the expressions of eNOS/sGC/ROCKII/PKG proteins in the present of various inhibitors. The inhibitors used in this experiment include a NOS inhibitor L-NAME, a sGC inhibitor ODQ and a PKG inhibitor Rp-8-CPT-cGMPS. The effects of Pulmodil on the expressions of eNOS, sGCa and sGCβ proteins in the present (FIG. 11(B)) or absent (FIG. 11(A)) U46619 is also determined. As shown, Pulmodil-induced the expressions of eNOS and sGC are inhibited by L-NAME and by ODQ, respectively. Inhibition of sGC and eNOS by ODQ indicates that decreased cGMP/PKG and increased ROCK down-regulates eNOS expression. Since ROCKII mediates down-regulation of eNOS, the inhibition of ROCKII caused by Pulmodil is in accordance to the increased eNOS expression. As shown in FIG. 11 (C), even the pretreatments with L-NAME to inhibit eNOS and with ODQ to inhibit sGC, Pulmodil inhibits the ROCKII expression, indicating the cGMP-dependency not only through the activations of eNOS and sGC but also through the inhibition of PDE5A. As shown in FIG. 11 (C), a PKG inhibitor Rp-8-CPT-cGMPS blunts Pulmodil-induced reduction of ROCKII, indicates that the Pulmodil-induced inhibition on ROCK is associated with the dependency on PKG. As shown in FIG. 11 (C), Rp-8-CPT-cGMPS also blunts Pulmodil-induced enhance of PKG protein.

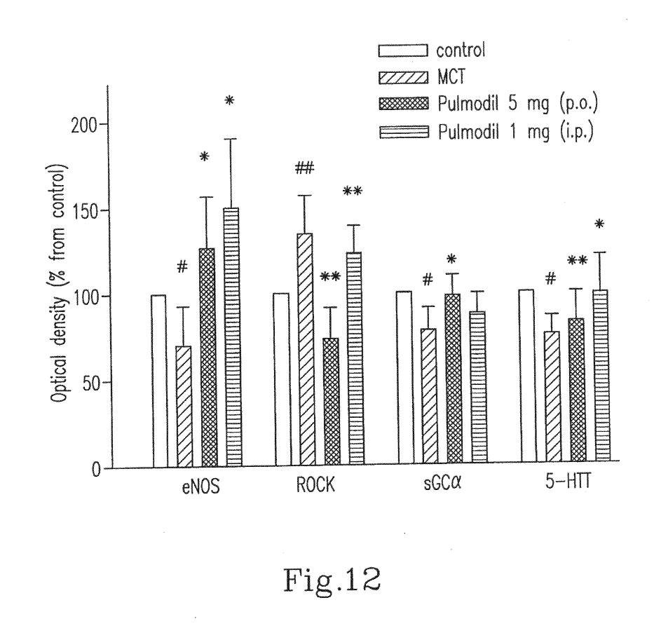

Please refer to FIG. 12, which shows the effects of Pulmodil on the monocrotaline (MCT)-induced changes in expressions of eNOS/sGC/5-HTT/ROCKII proteins. Western blotting analysis demonstrates that the expressions of eNOS and sGC are decreased and the expression of ROCK II is increased in lung tissues of MCT-treated (60 mg/kg, i.p.) rats. Pulmodil reverses the expressions of these proteins.

Based on the above protein assays, Pulmodil, a theophylline-based PDE5 inhibitor, obviously inhibits U46619-induced PDE5A and increase cGMP. Thus, a combination of the enhancing activities of cGMP, eNOS, sGC and PKG and the inhibiting activity of ROCK caused by inhibiting PDE5A is suggested to provide optimal achievement in inhibiting a medial thickness, an endothelium dysfunction or a vascular obstruction of the pulmonary artery. According to the recent studies regarding rho-kinase inhibitor, in addition to the effect on the vascular smooth muscle cells, it is also proved to be effective in relieving the cardiovascular disease. One example for such rho-kinase inhibitor is “Fasudil”, which is widely used in the therapy of cardiovascular disease. Therefore, one skilled in the art could reasonably anticipate that Pulmodil provided in the present invention could be applied to cardiovascular disease since Pulmodil is also a kind of rho-kinase inhibitors.

11. Histological Examination of Lung and Heart of MCT-Treated Rats:

For light microscopy studies, right and left pulmonary arteries, as well as intrapulmonary arteries, of six rats from each treatment group are isolated 3 weeks after MCT (60 mg/kg, i.p.) or vehicle administration. The samples are fixed in paraformaldehyde, soaked in formalin, dehydrated through graded alcohols, and embedded in paraffin wax for subsequent sectioning. The samples embedded in paraffin wax are cut into 5-μm-thick sections and subjected to hematoxylin-eosin (HE) staining before light microscopic examination.

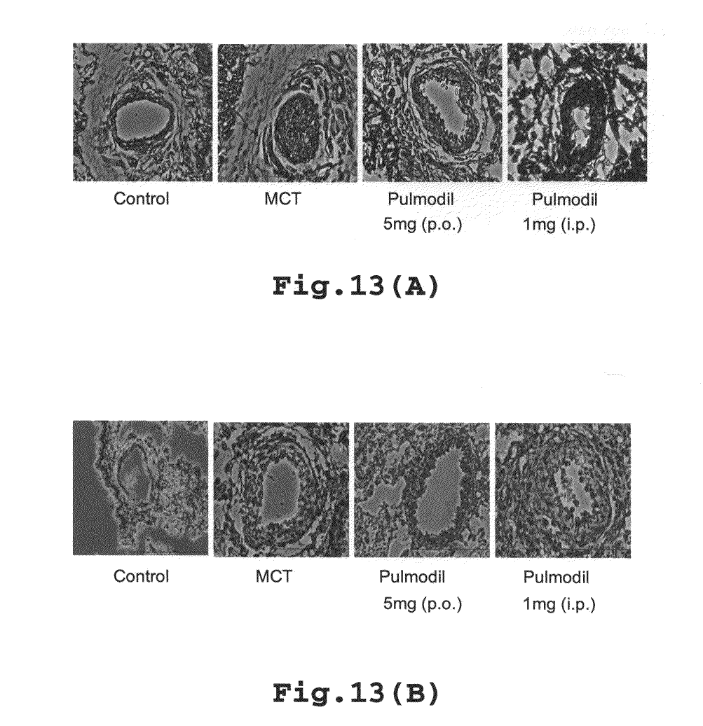

Please refer to FIGS. 13 (A) and (B), which shows the effects of Pulmodil on the morphology of pulmonary artery (A) and right ventricle (B) in the MCT-treated rats. As shown in the stained sections and the corresponding data in Table 6, the ratio of right ventricule (RV)/left ventricule (LV)+septum (S), and muscularization of distal pulmonary artery are significantly lower in MCT-treated rats given by Pulmodil than in those given by vehicle.

As shown in FIG. 13 (A), under the chronic PAH induced by MCT, Pulmodil has obvious extension effect on the pulmonary arteries, and decreases the pulmonary artery thickness as well as the right ventricular hypertrophy. Although Pulmodil has slight difference between the effects on the U46619-induced PAH and on the MCT-induced PAH because of different pathogenesis, Pulmodil could relieve either acute or chronic PAH induced by U46619 or MCT, respectively, and thus cause a significant decrease of the occurrence of right ventricular hypertrophy or failure.

TABLE 6

| | | Pulmonary artery |
|---|---|---|
| | RV/LV + S (%) | wall thickness (%) |
| Control | 21.4 ± 0.4 | 26.9 ± 2.7 |
| MCT | 44.8 ± 1.0## | 59.9 ± 12.7# |
| Pulmodil (5 mg, p.o.) + | 31.5 ± 0.7* | 30.7 ± 8.6* |
| MCT | | |
| Pulmodil (1 mg, i.p.) + | 35.8 ± 1.0* | 40.8 ± 3.5 |
| MCT | | |

### Embodiment 4

### Hypoxia Model

In in vivo hypoxic experiments, rats are divided into four groups:

Group 1. Normoxia, i.e. under normal growth condition;

Group 2: Hypoxia for 21 days;

Group 3: Hypoxia+Pulmodil for 21 days;

Group 4, Hypoxia+sidenafil for 21 days.

All rats are on a 12-h light/12-h dark cycle at 25±1° C. and are provided with food and water. Some rats are housed in standard normoxic conditions as control Group 1 and Groups 2-4 are continuously housed in a hypoxic chamber (10% O₂) for 21 days, except for a 30-min interval each day when the chamber is cleaned. The hypoxic gas mixture was prepared from N₂ (gas cylinders) and compressed air.

In in vitro experiments, isolated rat pulmonary artery is grown under normoxia (20% O₂) and hypoxia (1% O₂). To achieve hypoxia, a pre-analyzed gas mixture (95% N₂-5% CO₂) is infused into a CO₂ incubator (Class II series, Thermo Form a, USA). The temperature is maintained at 37° C. during experiments for 24 hrs.

1. Pulmonary Arterial Pressure Measurements.

The measurements of heart rate and MPAP of male Wistar rats, 10 weeks-old, are described in detail in embodiment 3, wherein the rats are anesthetized with urethane (1.25 g/Kg). Please refer to Table 7, which shows the effects of Pulmodil and sidenafil on the artery pressure under the Hypoxia condition. MPAP of normoxic and hypoxia-treated rats are 12.9±0.9 mmHg and 26.5±0.6 mmHg (n=6), respectively. Long-term treatment with Pulmodil and sidenafil (5 mg kg⁻¹ day⁻¹, p.o. for 21 days) on hypoxia-treated rats, the MPAP is markedly attenuated to 16.9±1.1 mmHg and 19.8±0.7 mmHg (n=6) at 21 day. As to other measurements, Pulmodil and sidenafil do not significantly affect the heart rate, mean artery pressure (MAP) and heart weight/total weight of each group of rats. The values in Table 7 is presented by mean value±standard error, wherein * indicates P<0.05, compared to Normoxia group 1 and # indicates P<0.05, compared to Hypoxia group 2.

TABLE 7

| | Group 1 | Group 2 | Group 3 | Group 4 |
|---|---|---|---|---|
| Total weight (g) | 421.4 ± 7.7 | 237.8 ± 8.3* | 288.8 ± 7.9^{#} | 275 ± 10.4^{#} |
| Heart rate (beats/min) | 375.8 ± 14.4 | 380.7 ± 12 | 342.1 ± 15.9 | 352.2 ± 36.4 |
| MPAP (mmHg) | 12.9 ± 0.9 | 26.5 ± 0.6* | 16.9 ± 1.1^{#} | 19.8 ± 0.7^{#} |
| MAP (mmHg) | 91.8 ± 1.3 | 90.4 ± 3.7 | 89.7 ± 2.9 | 93.6 ± 3.1 |
| Heart/Total weight (mg/g) | 3.6 ± 0.1 | 5.1 ± 0.2* | 4.1 ± 0.3^{#} | 4.2 ± 0.2^{#} |

As shown in Table 7, it could be known that Pulmodil could specifically relax the pulmonary arteries and thereby attenuate the pulmonary arterial pressure and reduce the occurrence of the pulmonary arterial diseases.

2. Histological Examination of Lung and Heart.

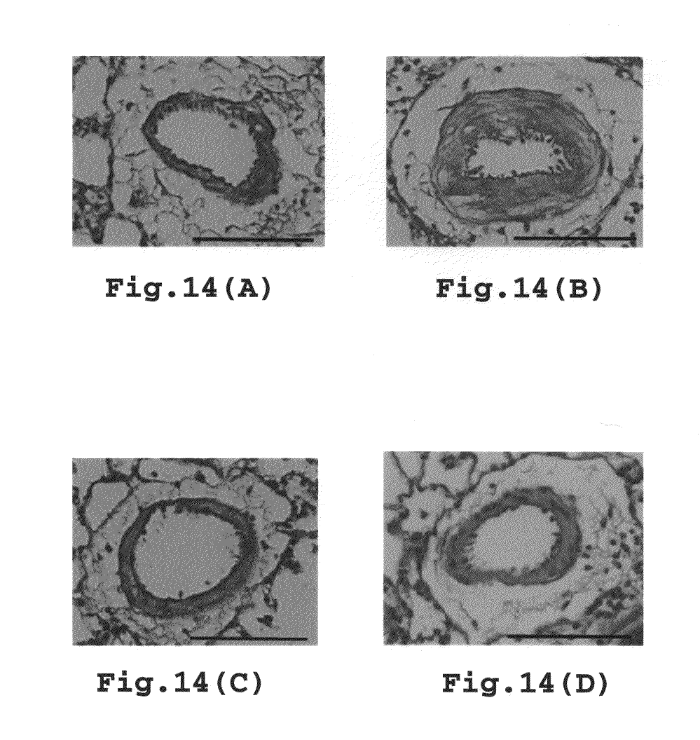

Pulmonary artery wall thickness is detected on day 0 and day 21, following left lung resection. Please refer to FIGS. 14 (A) to (D), which show the histological sections of pulmonary arteries of different groups of rats in Embodiment 4. As shown, small pulmonary arterial morphology is highly improved in Pulmodil-treated rats.

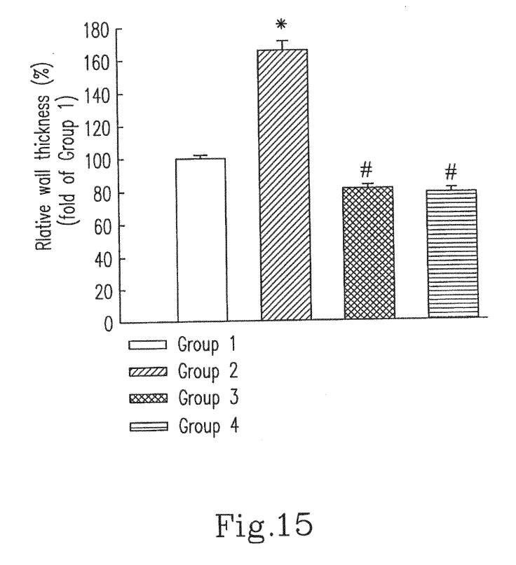

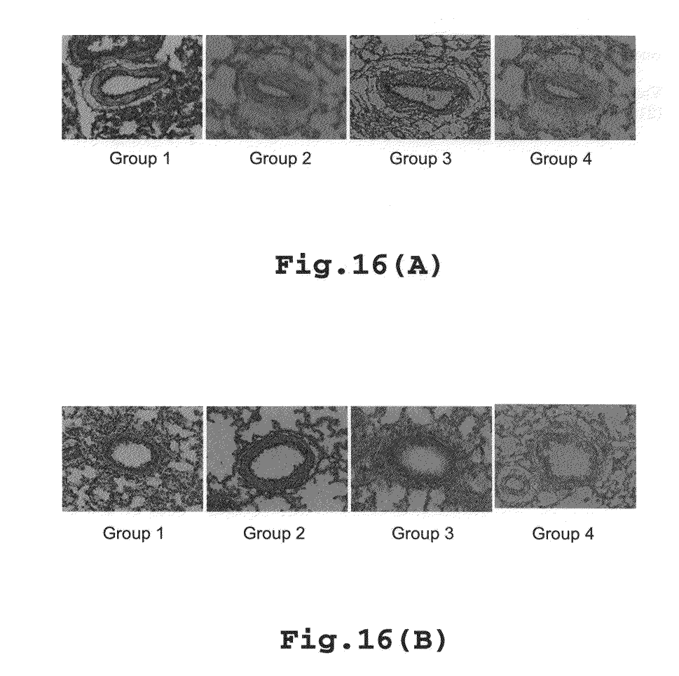

As shown in FIG. 14, sections stained with HE indicates that muscularizations of distal pulmonary artery are significantly lower in hypoxic rats given by Pulmodil and sidenafil than in those given by vehicle. As shown in FIG. 15, Pulmodil (5 mg kg⁻¹ day⁻¹, p.o. for 21 days) inhibits hypoxia-induced increase of relative wall thickness of pulmonary artery from 165.8±5.4% to 81.3±2.3% (P<0.05), compared to normoxia control (100%), and sidenafil has a similar effect as Pulmodil. Please refer to FIGS. 16 (A) and (B), which show the morphometricly immunostaining for eNOS and VEGF in pulmonary arteries. As shown in FIG. 16 (A), a marked increase of eNOS is located mainly in endothelium of pulmonary artery and correlated with media thickening. As shown in FIG. 16 (B), VEGF-immunostaining is also major located in endothelium and more significantly in smooth muscle, in comparison with arterial section (as a control) without immunostaining. In long term chronic hypoxia, repeated eNOS activation by Pulmodil in endothelium, opposing to ROCK inhibition, could prolong the duration of vascular relaxation and reduction of vascular resistance through eNOS/sGC/PKG pathway.

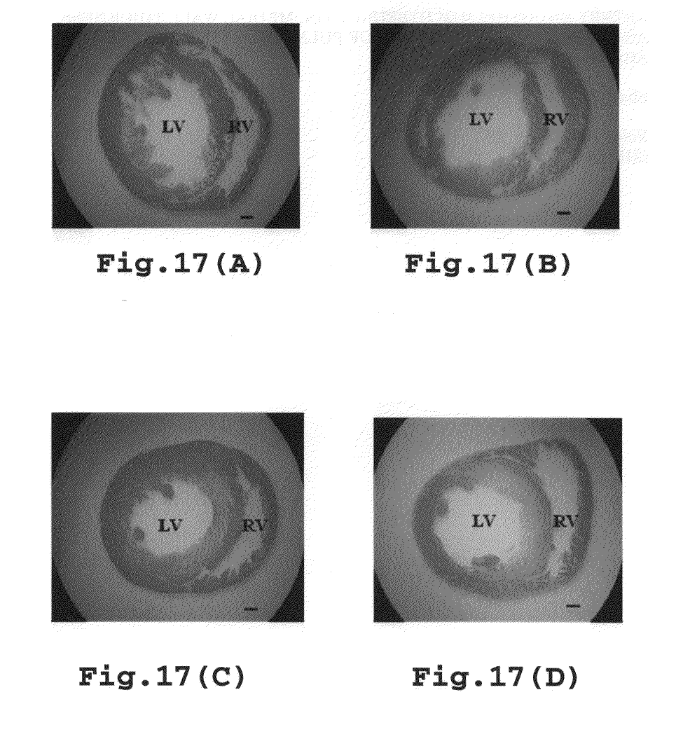

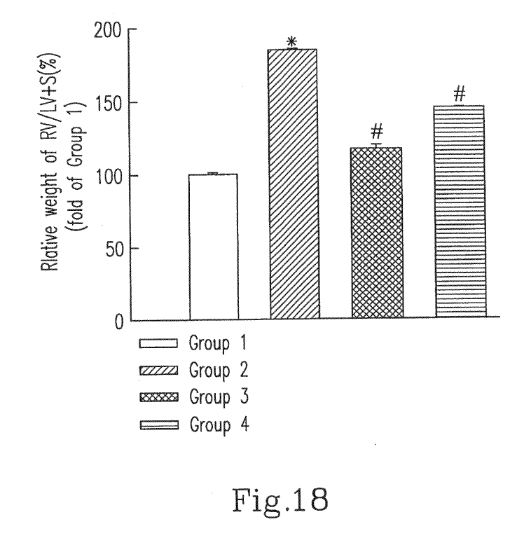

FIGS. 17 (A) to (D) show the histological sections of heart tissues of different groups of rats. In FIG. 18, the right heart index is represented by the relative weight of Right ventricle (RV)/[left ventricle (LV)+ intraventricular septum (S)] ratio, which is set 100% in normoxia group (group 1). During hypoxia condition, Pulmodil and Sildenafil could reduce the ratio from 184.6±0.7% (hypoxia group, group 2) to 117.4±2.6% (group 3) and 145.3±0.4% (P<0.05), compared with those from normoxia group (100%).

3. Western Blotting Analysis.

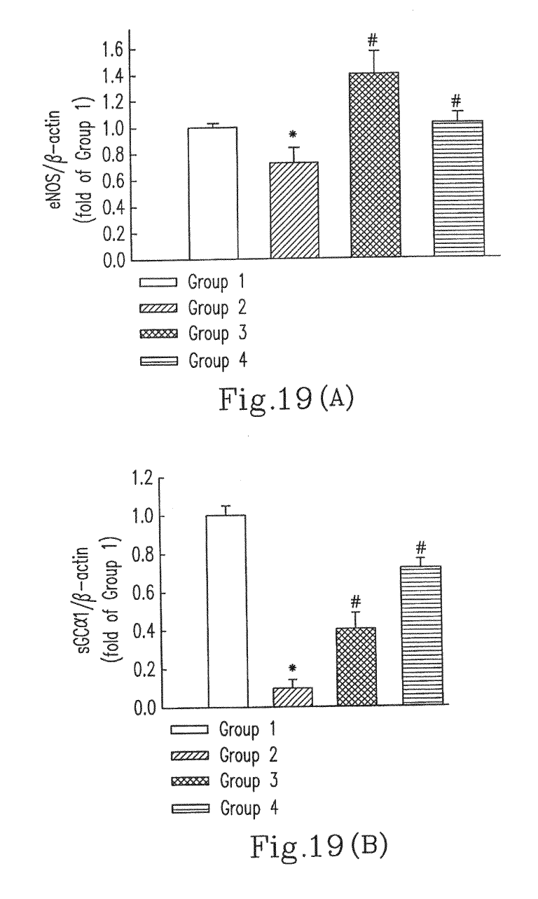

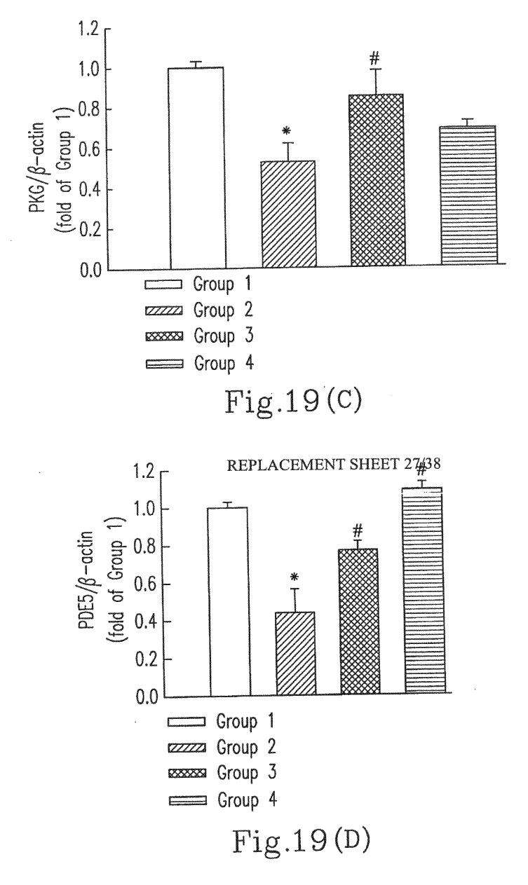

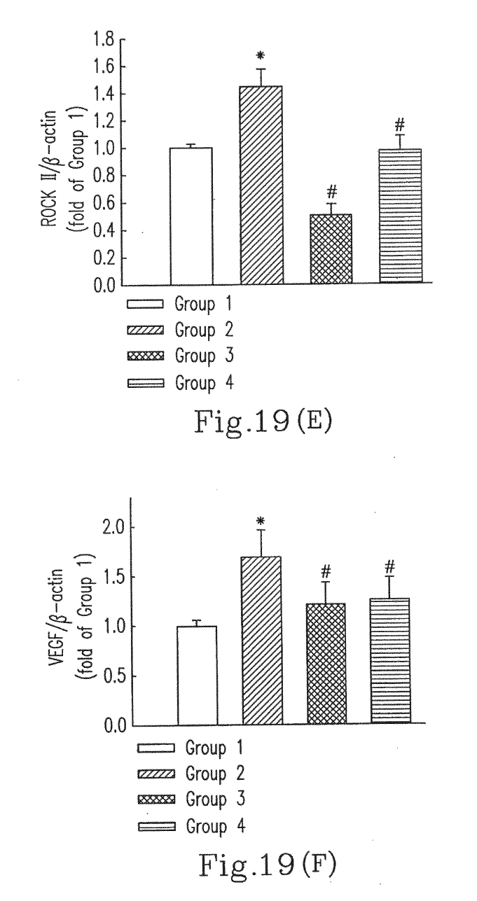

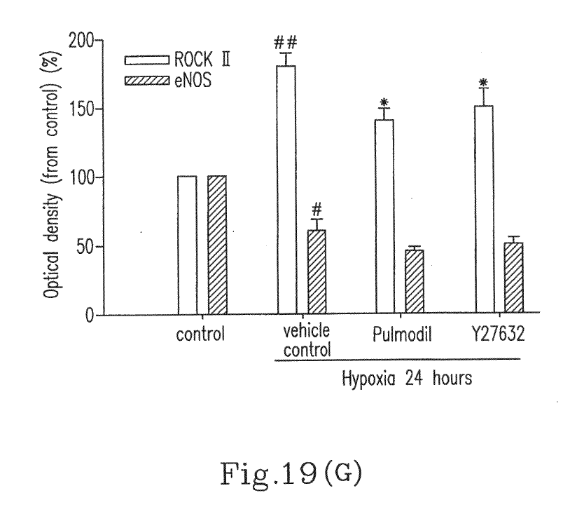

Please refer to FIGS. 19. (A) to (D), which show the effects of Pulmodil and Sildenafil on hypoxia-induced changes in eNOS, sGC, PKG and PDE5A protein expressions in rat lung tissue. Western blotting analysis demonstrates that eNOS, sGC and PKG are decreased in lung tissue of hypoxia-treated rats. In contrast, PDE-5A is increased by hypoxia in lung tissue. However, Pulmodil reverses the expressions of these proteins in hypoxia. Comparing with Sildenafil, Pulmodil is more effective in increasing the expressions of eNOS and PKG and less effective in increasing the expression of sGC.

Please refer to FIGS. 19 (E) and (F), expressions of VEGF and ROCKII are increased in chronic hypoxia for 21 days. Treatment with Pulmodil during this period prevents from hypoxia-induced increase of VEGF and ROCKII expression, compared to non-treatments. Comparing with Sildenafil, Pulmodil is more effective in reducing ROCKII expression and has similar effects on VEGF. ROCK is suggested to be related to the expression of VEGF, which is associated pulmonary artery remodeling and vascular hyperplasia in hypoxia. Pulmodil inhibits ROCK and VEGF, which indicates that Pulmodil has anti-hyperplasia activities including activities of inhibiting a medial thickness, an endothelium dysfunction or a vascular obstruction of the pulmonary artery.

Further, as shown in FIG. 19 (G), under short-term (24 hr) hypoxia condition, the expression of eNOS is decreased and that of ROCKII is increased. Treatments with Pulmodil and Y27632 during this period prevent from hypoxia-induced increase of ROCKII expression, compared to non-treatments.

Based on the chronic hypoxia experiments, it is indicated that Pulmodil inhibits hypoxia-induced pulmonary artery hypertension (PAH) in rats through increase of eNOS/sGC/PKG and decrease of ROCK/VEGF expressions. Further, Pulmodil is more effective in eNOS than Sildenafil.

### Embodiment 5

### Sublingual Administration Model

In addition to oral administration and intravenous infusion, it is proved in this invention that Pulmodil is effective by sublingual administration as well. The MCT-treated rats are used as samples for proving the effects of sublingual administration.

Adult male Wistar rats, weighted 200-250 g, are given a single subcutaneous injection of monocrotalin (MCT, 60 mg/kg) or vehicle and allowed 21 days to develop PAH. Sublinger preparation of Pulmodil (2.5 mg/kg/day/25 μL propylene glycol) is coated by a micropipette on sublingual cavity of rats. Rat mouth in non-analgesia condition is opened to allow application of Pulmodil with micropipette within 1 minute.

In the following relevant experiments, rats are divided into four groups:

Group 1: Control;

Group 2: MCT induction;

Group 3: sublingual administration of Pulmodil after the MCT induction; and

Group 4, sublingual administration of sidenafil after the MCT induction.

1. Pulmonary Arterial Pressure Measurements.

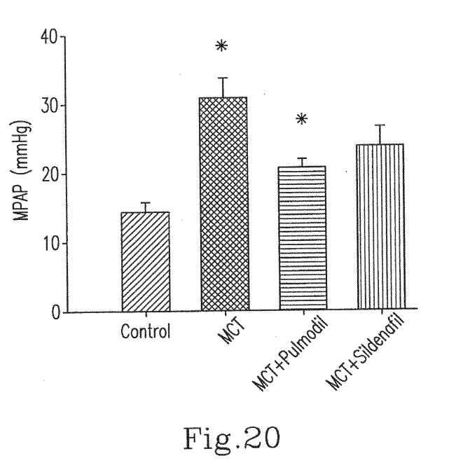

Please refer to FIG. 20, which shows measurements of MPAP of different groups of rat in Embodiment 5. In the long-term treatment with sublingual Pulmodil (2.5 mg kg⁻¹ day⁻¹) or sildenafil (2.5 mg kg⁻¹ day⁻¹ p.o.) for 21 days on MCT-treated rats, the developed PAH is markedly attenuated on the last day, respectively, in comparison with MCT-treated rats (Group 2). This result is similar to that by the oral administration and intravenous infusion in Table 4.

In the four groups, medial thickness (μm) and medial wall area (calculated as the area between the internal elastic lamina and the adventitia) of the muscular layer of pulmonary arteries are determined using an Eclipse E600 microscope (Nikon, Champigny-sur-Marne, France) coupled to a color video camera (Sony, Paris, France). Measurements are obtained with Histolab software (Microvision instruments, Evry, France). For each animal, medial thickness and medial wall area determination are taken as the average of three measurements.

2. Histological Examination of Lung and Heart.

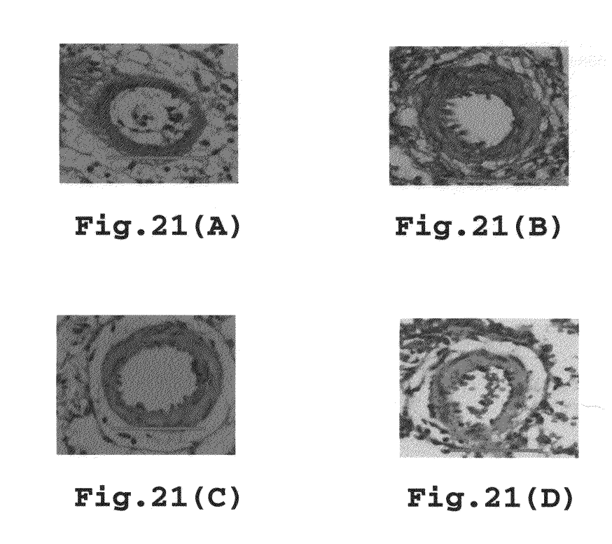

As described above, the pulmonary artery wall thickness is detected on day 0 and day 21. Please refer to FIGS. 21 (A) to (D), which show the histological sections of pulmonary arteries of different groups of rats. As shown in FIG. 21 (C), small pulmonary arterial morphology is highly improved in Pulmodil-treated rats.

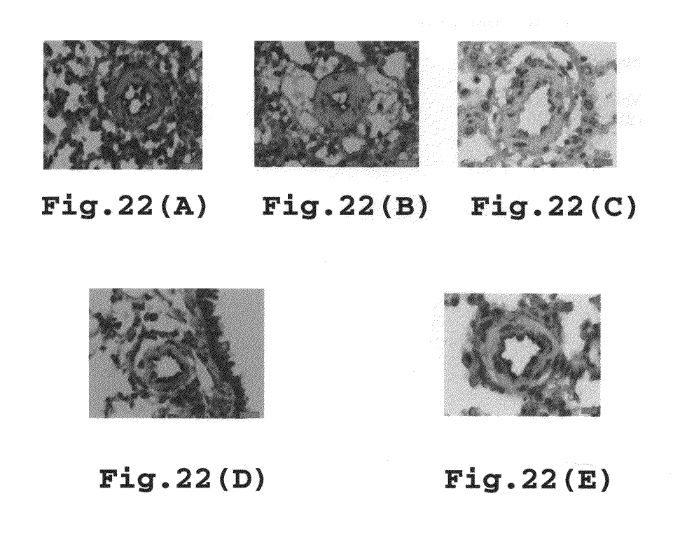

Please refer to FIGS. 22 (A) to (E), which show the morphometricly immunostaining for eNOS in different groups of rat pulmonary arteries in Embodiment 5. As shown, treatments with Pulmodil and sildenafil restore the decay of eNOS immunostaining, respectively, which is associated with the media thickening.

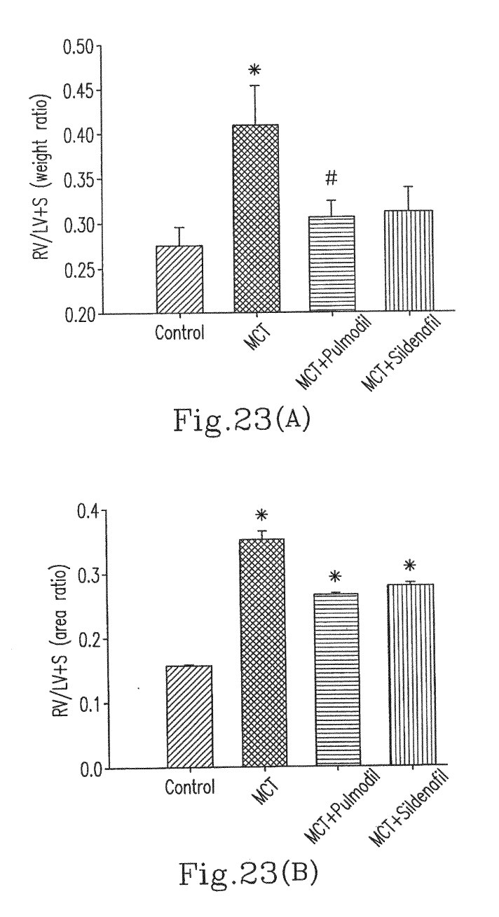

Please refer to FIGS. 23 (A) and (B), which show the right heart index of different groups of rats. In FIG. 23 (A), the right heart index is represented by the relative weight of Right ventricle (RV)/[left ventricle (LV)+ intraventricular septum (S)] ratio, and both Pulmodil and Sildenafil could markedly restore the MCT-induced increase of the right heart index, compared with those from control group (100%). In FIG. 23 (B), the right heart index is represented by the relative area of RV/LV+S, and both Pulmodil and Sildenafil could significantly reduce the MCT-induced increase of the right heart index, compared with those from control group (100%).

3. Western Blotting Analysis.

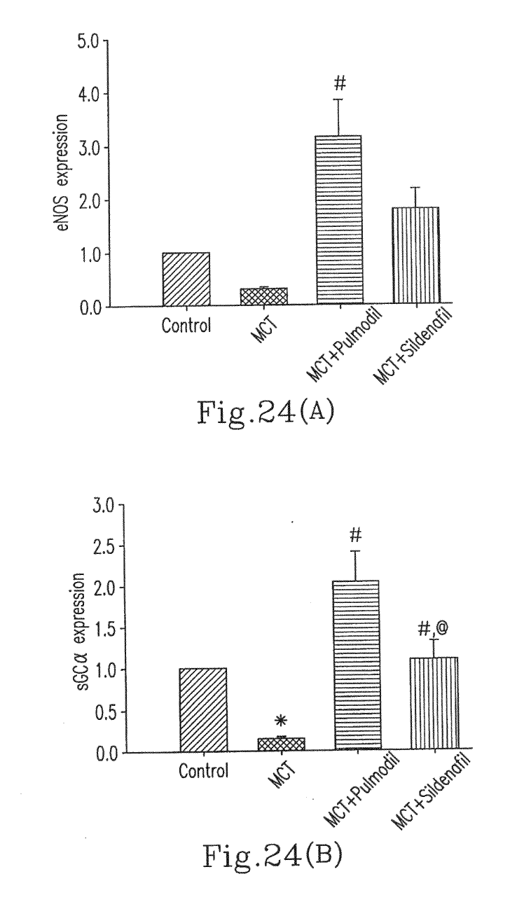

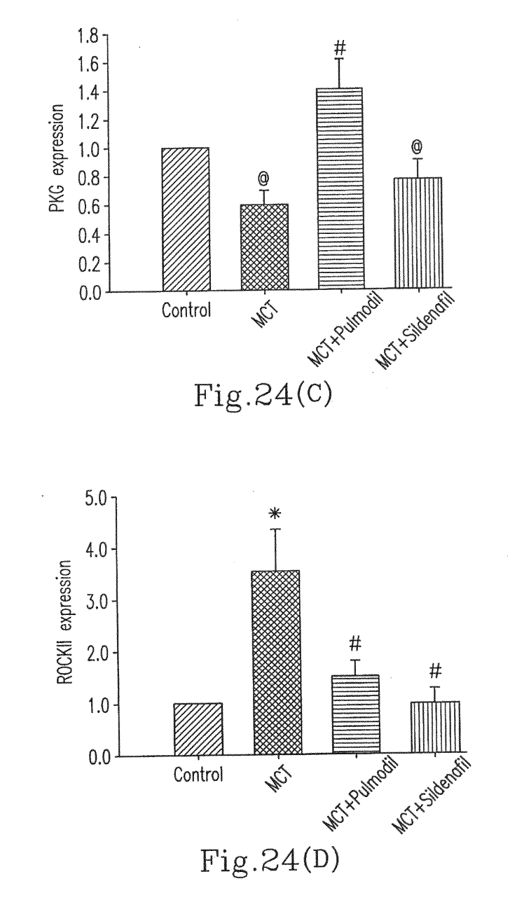

Please refer to FIGS. 24 (A) to (D), which show the effects of Pulmodil and Sildenafil on MCT-induced changes in eNOS, sGCa and PKG protein expressions in rat lung tissue. Western blotting analysis demonstrates that the markedly decreased eNOS and sGCa expressions in lung tissue of MCT-treated rats are increased greatly by Pulmodil and sildenafil. In contrast, as shown in FIGS. 24 (C), Pulmodil certainly increases PKG in MCT-treated rats but sildenafil insignificantly increases PKG therein.

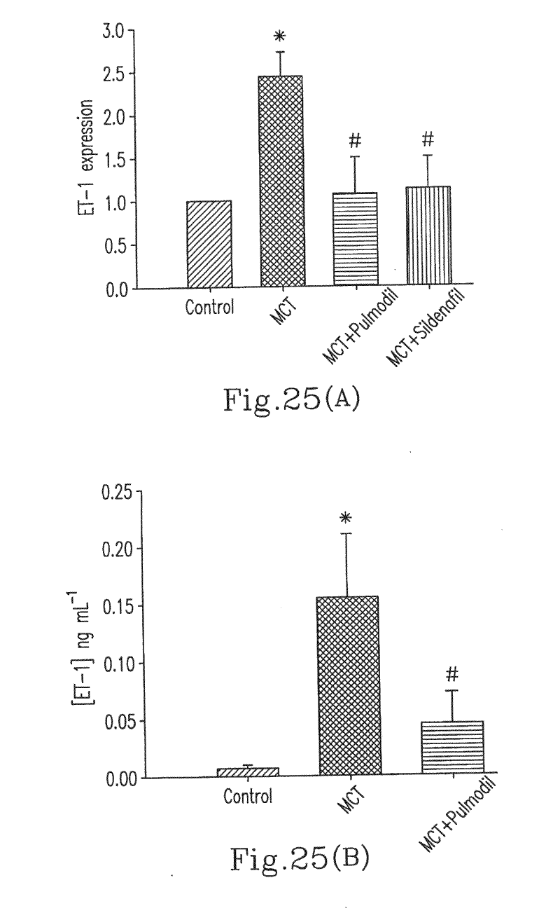

Please refer to FIG. 24 (D) and FIG. 25 (A), which show the effects of Pulmodil and Sildenafil on MCT-induced changes in ROCKII and ET-1 protein expressions in rat lung tissue. In the development of severe PAH, altered production of various endothelial vasoactive mediators, such as NO, prostacyclin, endothelin-1, serotonin, chemokines and thromboxane, have been increasingly recognized in patients with PAH. Among them, decrease of endothelial NO and increase of ET-1, attributing to pulmonary artery vasoconstriction and proliferation, are mainly involved in the pathogenesis of MCT-induced endothelial dysfunction and PAH. Therefore, ET-1 could be used as an index of MCT-induced PAH. FIG. 24 (D) and FIG. 25 (A), the expressions of ROCKII and ET-1 are increased to 350±80% and 244±28%, respectively in MCT-treated rats. Compared with non-treated group, treatments with Pulmodil and sildenafil decrease the MCT-induced increase of ROCKII to 150±30% and 100.7±3%, respectively, and that of ET-1 to 107±42% and 112±37%. Based on the above results, it could be known that Pulmodil and sildenafil given by the sublingual administration are also effective in MCT-induced PAH.

Comparing with sublingual Sildenafil, sublingual Pulmodil is more effective in reducing ROCKII expression and has similar effects on ET-1.

4. Plasma Concentration of Pulmodil and et-1.

Plasma concentration of Pulmodil (lower range: 0.1-20 ng/ml and higher range: 10-5000 ng/ml) in rats received 3.6 mg/kg Pulmodil, dissolved in propylene glycol and sublingually administered with a micropipet, is measured by an LCpMS/MS method. The plasma concentration of ET-1 is determined using an enzyme immunoassay kit, according to the commercial guidance of manufacturer (Biomedica Group, Wien, Austria). Cardiac puncture through needle (19G) on rats is performed to obtain blood and followed by centrifugation to have plasma sample. Plasma samples (0.8-1.0 ml) are acidified with 0.6% trifluoroacetic acid and centrifuged (2000 g, 48 degree C. for 15 mins).

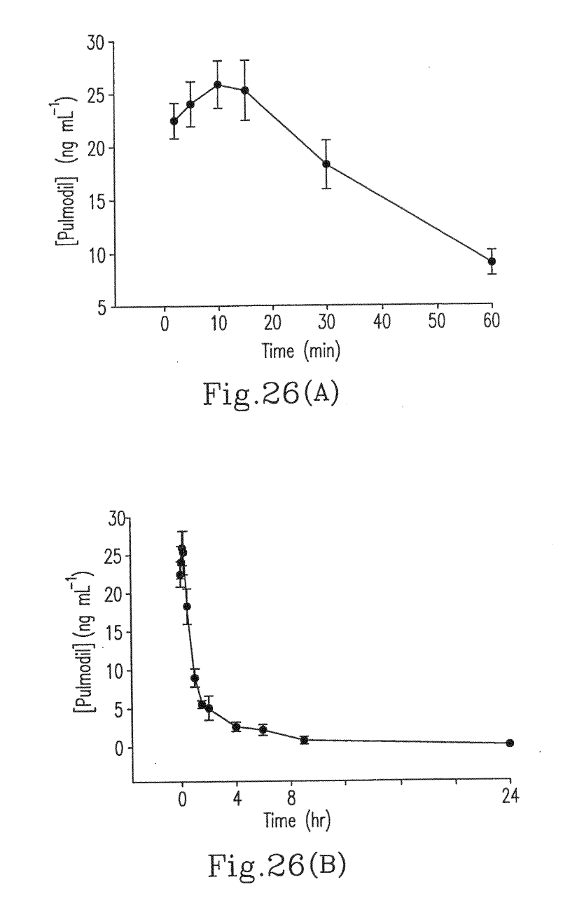

Please refer to FIGS. 26 (A) and (B), which show the concentration changes of Pulmodil in plasma in different groups of rats in Embodiment 5. As shown in FIG. 26 (A), the maximal plasma concentration of Pulmodil appears at 0.25 hr after the sublingual administration. Further, as shown in FIG. 26 (B), the determined Pulmodil in plasma is sustained for 6 hrs in rats. Sample's concentration below 0.1 ng/mL is reported as blq (below lower limit of quantification).

Please refer to FIG. 25 (B), which shows the effect of Pulmodil on the plasma concentration of ET-1 in different groups of rats in Embodiment 5. Pulmodil in [MCT+Pulmodil] group displays lower level of ET-1 (0.05±0.02 ng/mL), compared to control group (0.007±0.002 ng/mL) and MCT-treated group (0.16±0.06 ng/mL), respectively. That is to say, Pulmodil decreases concentration of ET-1 in plasma of MCT-treated rats.

The merit of sublingual administration of Pulmodil is to increase the bioavailability, compared to oral administration. Further, sublingual administration of Pulmodil is certainly absorbed into plasma and could indicate the pharmacologic profile of long term administration of Pulmodil to prevent from MCT-induced PAR The long term effect for at least three weeks reflects the pharmacologic feature processed by the specific structure of Pulmodil.

Based on all of the above, it could be known that the inhibition of Pulmodil on drug-induced PAH is achieved by inhibiting PDE, enhancing NO/cGMP and increasing the K⁺-channel opening activity. As to the Hypoxia-induced PAH, the prevention effects on a pulmonary artery endothelium dysfunction, a thickened pulmonary artery medial wall, a vascular obstruction and a right ventricular hypertrophy is achieved by inhibiting ROCK/VEGF and activating eNOS/PKG. Such effect of increasing the activity of eNOS and thereby releasing NO is similar to that of statin drugs. Further, the various administration routs, e.g. an oral administration, an intravenous injection, a subcutaneous injection, an intraperitoneal injection, an intramuscular injection and a sublingual administration, increase the bioavailability. Therefore, regarding various drugs-induced PAH, the applications of Pulmodil in reducing a pulmonary artery endothelium dysfunction, a thickened pulmonary artery medial wall and a vascular obstruction are proved, and have wide-range effects than other drugs for treating PAH. Pulmodil provided in the present invention could be applied to acute or chronic PAH induced asthma, breathing disease, chronic obstructive pulmonary disease (COPD), anaphylaxis, pulmonary fibrosis, pulmonary emboli, or right ventricular hypertrophy or failure, for effectively reducing the death rate of the patients.

While the invention has been described in terms of what is presently considered to be the most practical and preferred embodiments, it is to be understood that the invention needs not be limited to the disclose embodiments. Therefore, it is intended to cover various modifications and similar arrangements included within the spirit and scope of the appended claims, which are to be accorded with the broadest interpretation so as to encompass all such modifications and similar structures.

What is claimed is:

1. A pharmaceutical composition for treating one of a cardiovascular disease and a pulmonary artery disease, comprising one of a first compound having a Formula I: 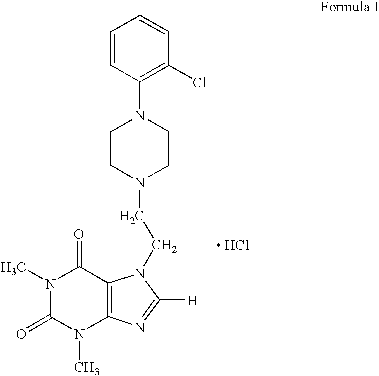
and a second compound having a Formula II:

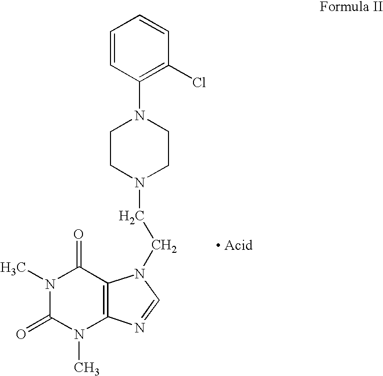

2. The pharmaceutical composition according to claim 1, wherein the pulmonary artery disease comprises one selected from a group consisting of a pulmonary artery endothelium dysfunction, a thickened pulmonary artery medial wall, and a vascular obstruction.

3. The pharmaceutical composition according to claim 1, wherein the HCl of Formula I and the acid of Formula II are derived from at least one of a xanthine and a piperazine.

4. The pharmaceutical composition according to claim 1, wherein the acid is one of an organic acid and an inorganic acid.

5. The pharmaceutical composition according to claim 4, wherein the organic acid comprises one selected from a group consisting of a citric acid, a maleinic acid, a fumaric acid, a tartaric acid, an oleic acid, a stearic acid, a benzenesulphonic acid, an ethyl benzenesulphonic acid, a benzoic acid, a succinic acid, a mesylic acid, a dimesylic acid, an acetic acid, a propionic acid, a pentanoic acid and an aspartic acid.

6. The pharmaceutical composition according to claim 4, wherein the inorganic acid comprises one selected from a group consisting of a hydrochloride, a sulfuric acid, a phosphoric acid, a boric acid and a dihydrochloride.

7. The pharmaceutical composition according to claim 1, further comprising at least one of a pharmaceutically acceptable carrier and an excipient.

8. The pharmaceutical composition according to claim 1, wherein the second compound is a 7-[2-[4-(2-chlorophenyl)piperazinyl]-ethyl]-1,3-dimethylxanthine.Citric acid.

9. A method for relieving a symptom including one selected from a group consisting of a pulmonary artery endothelium dysfunction, a thickened pulmonary artery medial wall, and a vascular obstruction in a mammalian subject in need thereof, comprising: administering to the mammalian subject a pharmaceutically effective amount of a pharmaceutical composition including one of a first compound having a Formula I: 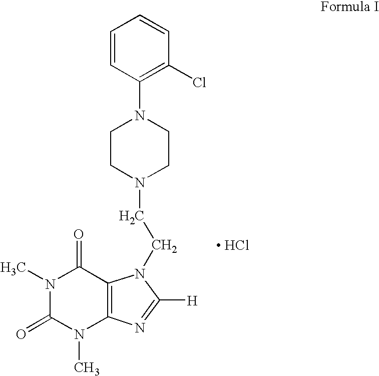
and a second compound having a Formula II:

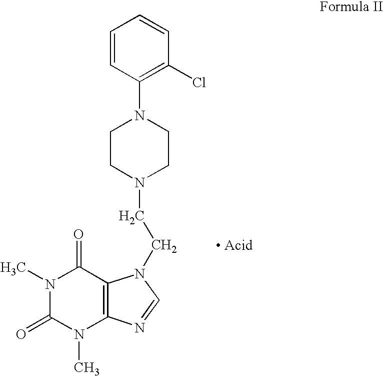

10. The method according to claim 9, wherein the mammalian subject is a human.

11. The method according to claim 9, wherein the administration comprises one selected from a group consisting of an oral administration, an intravenous injection, a subcutaneous injection, an intraperitoneal injection, an intramuscular injection and a sublingual administration.

12. The method according to claim 9, wherein the pharmaceutical composition further comprises at least one of a pharmaceutically acceptable carrier and an excipient.

13. The method according to claim 9, wherein the second compound is a 7-[2-[4-(2-chlorophenyl)piperazinyl]-ethyl]-1,3-dimethylxanthine-Citric acid.

14. A method for treating a cardiovascular disease in a mammalian subject in need thereof, comprising: administering to the mammalian subject a pharmaceutically effective amount of a substrate including one of a first compound having a Formula I: 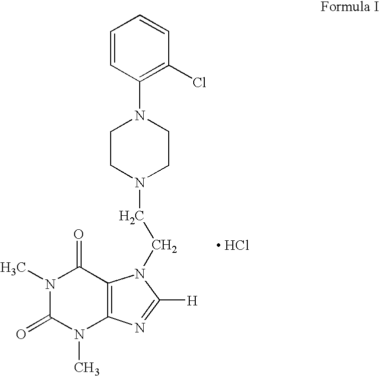
and a second compound having a Formula II:

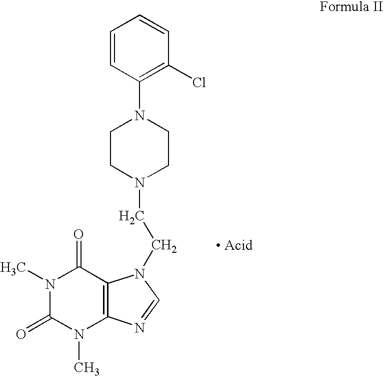

15. The method according to claim 14, wherein the administration comprises one selected from a group consisting of an oral administration, an intravenous injection, an subcutaneous injection, an intraperitoneal injection, an intramuscular injection and a sublingual administration.

16. The method according to claim 14, wherein the second compound is a 7-[2-[4-(2-chlorophenyl)piperazinyl]-ethyl]-1,3-dimethylxanthine.Citric acid.
